SEQUENCE LISTING (56 sequences)

SEQ ID NO: 1 - DNA, 25 bp, synthetic construct
  misc_feature 1..25: note = Description of Artificial Sequence: Synthetic primer
  source 1..25: mol_type = other DNA
atggcttctctattatcttctcgtc

SEQ ID NO: 2 - DNA, 24 bp, synthetic construct
  misc_feature 1..24: note = Description of Artificial Sequence: Synthetic primer
  source 1..24: mol_type = other DNA
tcatgggaagatagcattaacaaa

SEQ ID NO: 3 - DNA, 28 bp, synthetic construct
  misc_feature 1..28: note = Description of Artificial Sequence: Synthetic primer
  source 1..28: mol_type = other DNA
atggcttctctattatcttctcgtctcc

SEQ ID NO: 4 - DNA, 25 bp, synthetic construct
  misc_feature 1..25: note = Description of Artificial Sequence: Synthetic primer
  source 1..25: mol_type = other DNA
ttaaatagaatctgccacataaatg

SEQ ID NO: 5 - DNA, 26 bp, synthetic construct
  misc_feature 1..26: note = Description of Artificial Sequence: Synthetic primer
  source 1..26: mol_type = other DNA
atggatgctctcttcgtcaattcctc

SEQ ID NO: 6 - DNA, 23 bp, synthetic construct
  misc_feature 1..23: note = Description of Artificial Sequence: Synthetic primer
  source 1..23: mol_type = other DNA
gcccttgttcaacttactacatt

SEQ ID NO: 7 - DNA, 25 bp, synthetic construct
  misc_feature 1..25: note = Description of Artificial Sequence: Synthetic primer
  source 1..25: mol_type = other DNA
atggatgctctgttcgtcaattcct

SEQ ID NO: 8 - DNA, 25 bp, synthetic construct
  misc_feature 1..25: note = Description of Artificial Sequence: Synthetic primer
  source 1..25: mol_type = other DNA
tcaacttactacattctgttgaccc

SEQ ID NO: 9 - DNA, 27 bp, synthetic construct
  misc_feature 1..27: note = Description of Artificial Sequence: Synthetic primer
  source 1..27: mol_type = other DNA
atggaagccactgcttctttctcctca

SEQ ID NO: 10 - DNA, 26 bp, synthetic construct
  misc_feature 1..26: note = Description of Artificial Sequence: Synthetic primer
  source 1..26: mol_type = other DNA
ttagttcttggccccaaaaccacgag

SEQ ID NO: 11 - DNA, 1116 bp, Nicotiana tabacum
  source 1..1116: mol_type = genomic DNA
atggcttctctattatcttctcgtctcccacgtcatctttcctctaataaaccggtactcccaccatcaagctccggttcaaatctccttcacaacttcacatataaaacccggttcgatcaatcccggttcaaatgctcagctggtggaacggggttcttcacgaagttgggtcgtttgctgaaagagaaagcaaagagcgacgtggagaaactgttctcaggattctcaaaaactcgagacaatttagcagttatagatgaactcctcctttactggaacctttctgacactgaccgtgttcttgatgaacttgaagaggttctgttggtgtctgattttggcccgaagattaccataaagattgtggagagcttgcgggaggatatatatggggggaaaatcaaatcaggaagtgagattaaaagtgctcttaagaagagtatcttggatctattgactagcaaggcacctaaaacagagctccgtctgggcttcaggaaaccatctgtgatcatgattgtgggcgtcaacggaggtgggaagacaacatctcttggaaagctggcaaatagattgaagaaagaaggggctaagatactattagcagctggtgatacatttagagcagctgctagtgatcagttagaaatttgggctgaaaggactggttgtgagatcgttgttgctgaaaaagagaaagctaaggcatcatcagttctttcgcaggctgttaaaagaggaaaggaagagggtttcgatattgttttatgcgacacatctggccgtctgcacacctactatagcttgatggaggaattggtggcatgcaaaaaagttgtcagtaaaattgttactggtgcacctaatgaaatcttgcttgtactggatggaactactggtttaaatatgcttccacaagcaagagagtttaacgatgttgttggagtcactggcttaatattgactaaacttgatggttctgctcgaggtggctgtgtggttagtgtggttgatgaacttggcattcctgtaaagtttgtaggtgttggggaaggtgtagatgacctccaaccgttcaatgctgaggaatttgttaatgctatcttcccatga

SEQ ID NO: 12 - protein, 371 aa, Nicotiana tabacum
MASLLSSRLPRHLSSNKPVLPPSSSGSNLLHNFTYKTRFDQSRFKCSAGGTGFFTKLGRLLKEKAKSDVEKLFSGFSKTRDNLAVIDELLLYWNLSDTDRVLDELEEVLLVSDFGPKITIKIVESLREDIYGGKIKSGSEIKSALKKSILDLLTSKAPKTELRLGFRKPSVIMIVGVNGGGKTTSLGKLANRLKKEGAKILLAAGDTFRAAASDQLEIWAERTGCEIVVAEKEKAKASSVLSQAVKRGKEEGFDIVLCDTSGRLHTYYSLMEELVACKKVVSKIVTGAPNEILLVLDGTTGLNMLPQAREFNDVVGVTGLILTKLDGSARGGCVVSVVDELGIPVKFVGVGEGVDDLQPFNAEEFVNAIFP

SEQ ID NO: 13 - DNA, 966 bp, Nicotiana tabacum
  source 1..966: mol_type = genomic DNA
atggcttctctattatcttctcgtctcccacatcatctttcctctaataaaccggtactcccaccatcaagctccggttcaaatctccttcacaacttcacatataaaacccggttcgatcaatcccggttcaaatgctcagctggtggaacggggttcttcacgaagttgggtcgtttgctgaaagagaaagcaaagagcgacgtggagaaactgttctcaggattctcaaaaactcgagacaatttagcagttatagatgaactcctcctttactggaacctttctgacactgaccgtgttcttgatgaacttgaagaggttctgttggtgtctgattttggcccgaagattaccataaagattgtggagagcttgcgggaggatatatatggggggaaaatcaaatcaggaagtgagattaaaagtgctcttaagaagagtatcttggatctattgactagcaaggcacctaaaacagagctccgtctgggcttcaggaaaccatctgtgatcatgattgtgggcgtcaacggaggtgggaagacaacatctcttggaaagctggcaaatagattgaagaaagaaggggctaagatactattagcagctggtgatacatttagagcagctgctagtgatcagttagaaatttgggctgaaaggactggttgtgagatcgttgttgctgaaaaagagaaagctaaggcatcatcagttctttcgcaggctgttaaaagaggaaaggaagagggtttcgatattgttttatgcgacacatctggccgtctgcacacctactatagcttgatggaggaattggtggcatgcaaaaaagttgtcagtaaaattgttactggtgcacctaataggagcagtatacaacaggaactaagattagcagcttgctactactctgaaaattcaacattcacaaataacatttatgtggcagattctatttaa

SEQ ID NO: 14 - protein, 320 aa, Nicotiana tabacum
MASLLSSRLPHHLSSNKPVLPPSSSGSNLLHNFTYKTRFDQSRFKCSAGGTGFFTKLGRLLKEKAKSDVEKLFSGFSKTRDNLAVIDELLLYWNLSDTDRVLDELEEVLLVSDFGPKITIKIVESLREDIYGGKIKSGSEIKSALKKSILDLLTSKAPKTELRLGFRKPSVIMIVGVNGGGKTTSLGKLANRLKKEGAKILLAAGDTFRAAASDQLEIWAERTGCEIVVAEKEKAKASSVLSQAVKRGKEEGFDIVLCDTSGRLHTYYSLMEELVACKKVVSKIVTGAPNRSSIQQELRLAACYYSENSTFTNNIYVADS

SEQ ID NO: 15 - DNA, 1121 bp, Nicotiana tabacum
  source 1..1121: mol_type = genomic DNA
atggatgctctcttcgtcaattcctctctctcccgcctcaaactcaaattctcccctcaattccctcccaccttctctcatcaaccttttatctgtctaaagaaactcggcaataagaacaatttatcagtatttgctacgcttcagaaccagcagcaacaagcagtcgaagcagcagaagacgaagaaccggtactgtttgaagattacgatgaggatgaaacgtacggagaagttaacaaaattatcggcagtcgagcaattgaacgtgggaaaggaatggagtacttgatagagtggaaagacgaacatgccccaacgtgggtcccctctgattacattgctaaagatgttgtggccgagtacgaaactccttggtggaatgcggctaaaaaggccgacgaatccgctcttagggaactcctagaaactgacgacgacagagatgtggacgcagtagatgaggatggacgtacggctttgctctttgtctcgggtctggggtccgagccgtgtgtcaagctgctagctgaagccggcgctgacgtggactatcgcgataggaatggcggcttgactgctctgcatatggcagccggctatgttaagccgggtgtcgccaagctgttaattgacctcggggcagaccccgaggtcgaggattatagaggacaaacgcctctgagcttggcgaggatgattttgaatcaaacgcctaaaggaaatccaatgcaattcgcgaggagattggggttagagaatgtggttaggattttggaggatgcgattttcgagtatgcaacagtggaggaaatattggagaagagagggaaaggcgaaaatgtggagtatttagtgaagtggaaggatggggaggataacgagtgggtcaaagcatggctgatatctgaagatttggtgagggattttgaggctggtttggaatatgcagtagcagattgtattcttgagaagagagaaggtgaggatgggaagggagaatacttggttaaatggactgatattgaggaagctacgtgggaacccgaagaaaatgttgacccccttcttatagaagattttgaaaagagtcaacagaatgtagtaagttgaacaagggc

SEQ ID NO: 16 - protein, 370 aa, Nicotiana tabacum
MDALFVNSSLSRLKLKFSPQFPPTFSHQPFICLKKLGNKNNLSVFATLQNQQQQAVEAAEDEEPVLFEDYDEDETYGEVNKIIGSRAIERGKGMEYLIEWKDEHAPTWVPSDYIAKDVVAEYETPWWNAAKKADESALRELLETDDDRDVDAVDEDGRTALLFVSGLGSEPCVKLLAEAGADVDYRDRNGGLTALHMAAGYVKPGVAKLLIDLGADPEVEDYRGQTPLSLARMILNQTPKGNPMQFARRLGLENVVRILEDAIFEYATVEEILEKRGKGENVEYLVKWKDGEDNEWVKAWLISEDLVRDFEAGLEYAVADCILEKREGEDGKGEYLVKWTDIEEATWEPEENVDPLLIEDFEKSQQNVVS

SEQ ID NO: 17 - DNA, 1113 bp, Nicotiana tabacum
  source 1..1113: mol_type = genomic DNA
atggatgctctgttcgtcaattcctctctctcccgcctcaaactcaaattctcccctcaattccctcccaccttctctcatcaaccttttatccgtctaaagaaactcggcaacaagaacaatttctcagtatttgctacgcttcagaaccagcagcaacaagcagtcgcagctgctgaagaggaagaaccggtactgtttgaagattacgatgaggatgaaacgtacggagaagttaacaaaatcatcggaagtagagcaattgaaggtgggaaaggaatggagtacttgatagagtggaaagacgaacatgccccaacatgggtcccctctgattacattgctaaagatgttgtggccgagtacgaaactccttggtggaatgccgctaaaaaggccgacgaatccgctcttaagaaatttctagaagctgacgacgacagagatgtggacgcagttgatgaggatggacgtacggctttgctctttgtctcgggtctggggtccgagccgtgtgtcaagctgctagctgaagctggcgctgacgtggactatcgcgataggaatggcggcttgacggctctgcacatggcagccggctatgttaagccgggtgtcgccaagctgttaattgacctcggggcagaccctgaggtcgaggattatagaggacaaacgcctctgagcttggcgaggatgattttgaatcaaacgcctaaaggaaacccaatgcaatttgctaggagattgggactagagaatgtggttaggatattggaggatgcgattttcgaatatgcaacagtggaggagatattggagaagagagggaaaggtgaaaatgtggagtatttagtcaagtggaaggatggggaggataatgaatgggtgaaagcatggctgataagtgaggatttggtgagagattttgaggctggtttggaatatgcagtggcagagtgtattcttgagaagagagaaggtgaggatgggaagggagaatatttggttaaatggactgatattgaggaagctacctgggaaccggaagaaaatgttgacccccttctaatagaagattttgaaaagggtcaacagaatgtagtaagttga

SEQ ID NO: 18 - protein, 370 aa, Nicotiana tabacum
MDALFVNSSLSRLKLKFSPQFPPTFSHQPFIRLKKLGNKNNFSVFATLQNQQQQAVAAAEEEEPVLFEDYDEDETYGEVNKIIGSRAIEGGKGMEYLIEWKDEHAPTWVPSDYIAKDVVAEYETPWWNAAKKADESALKKFLEADDDRDVDAVDEDGRTALLFVSGLGSEPCVKLLAEAGADVDYRDRNGGLTALHMAAGYVKPGVAKLLIDLGADPEVEDYRGQTPLSLARMILNQTPKGNPMQFARRLGLENVVRILEDAIFEYATVEEILEKRGKGENVEYLVKWKDGEDNEWVKAWLISEDLVRDFEAGLEYAVAECILEKREGEDGKGEYLVKWTDIEEATWEPEENVDPLLIEDFEKGQQNVVS

SEQ ID NO: 19 - DNA, 1692 bp, Nicotiana tabacum
  source 1..1692: mol_type = genomic DNA
atggaagccactgcttctttctcctcaactatgtcttcccaccatttctttccactttccaaagccaccctctcaacttctaaacttccattttctgggactggttcaactcattctctttcattttcttcaagaaactcattcactagggatacatggagggtgatcaattcaaggaatgtggttatttcaagaagagaaatgcgtggagttattagagctgagatgtttggacagctcactagtggacttgaatcagcttggaataagctcaaaggagaagaggttttgaccaaggaaaacattgtggaacctatgagagacatcaggagggctcttttggaagctgatgttagtctccctgttgtcagaaggtttgttcagtctgttagtgacgaagccgtggggactggcttgattcgaggagtaagaccagatcagcaactagttaaaattgtacgagacgagcttgtgaaactgatgggtggagaggtctctgaactggtatttgctaaatctggacccaccataatactattggccggtctacaaggtgttggaaagacaactgttagcgcaaagttagctttatatctaaagaagcagggtaagagttgcatgctgattgctggagacgtgtatagacctgctgctattgaccaacttgttattttgggtgaacaggttgatgtgcctgtttatgcagcaggaacagacgtaaaacctgcagaaatagcccgtcaaggattagaagaggccaaaagaaagaatgtagatgtagtcataatggatacagctggacgacttcagatagataaagctatgatggatgaattaaaagaggtgaaacgggtactgaaccccacagaggttttgcttgttgtggatgcaatgactggccaagaagctgcagctttggtcacaacattcaatctcgaaattggaattactggtgccattatgacaaagctagatggggattctaggggtggagcagctttaagtgtcaaggaggtatcaggaaagccaattaagctcgtaggaagaggtgaacgtatggaggaccttgaacctttctatcctgaccgcatggctggacgtattttagggatgggagatgttctgtcgtttgttgaaaaagcccaagaagttatgaaacaagaagatgcagaagatttgcagaagaagatcatgagtgcaaaatttgatttcaatgacttcctgaagcaaactcgtgcagttgctcaaatgggtaccatgtcccgcgttctcggaatgattcctggcatgggaaaggttactcctgcacaaattcgagaggcagagaagagcttaataataatggagtcaatgatagaagtcatgacaccagaggagaaggagaaaccagaactgttagcagaatctcctagtagaaggaaacgtattgctcaagagtccgggaaaactgagcagcaggtgagtcaacttgttgctcaactttttcaaatgcgtgtacgtatgaagaatttgatgggtgttatgcaaggtggttccatacctgcactgagtaatcttgaggaggcacttaaaactgaacagaaggctcctcctggtactgcaaggaggaagcgaaggtcagaatcaagaaagcaatttgcagactcgggatcaactagacctggccctcgtggttttggggccaagaactaa

SEQ ID NO: 20 - protein, 563 aa, Nicotiana tabacum
MEATASFSSTMSSHHFFPLSKATLSTSKLPFSGTGSTHSLSFSSRNSFTRDTWRVINSRNVVISRREMRGVIRAEMFGQLTSGLESAWNKLKGEEVLTKENIVEPMRDIRRALLEADVSLPVVRRFVQSVSDEAVGTGLIRGVRPDQQLVKIVRDELVKLMGGEVSELVFAKSGPTIILLAGLQGVGKTTVSAKLALYLKKQGKSCMLIAGDVYRPAAIDQLVILGEQVDVPVYAAGTDVKPAEIARQGLEEAKRKNVDVVIMDTAGRLQIDKAMMDELKEVKRVLNPTEVLLVVDAMTGQEAAALVTTFNLEIGITGAIMTKLDGDSRGGAALSVKEVSGKPIKLVGRGERMEDLEPFYPDRMAGRILGMGDVLSFVEKAQEVMKQEDAEDLQKKIMSAKFDFNDFLKQTRAVAQMGTMSRVLGMIPGMGKVTPAQIREAEKSLIIMESMIEVMTPEEKEKPELLAESPSRRKRIAQESGKTEQQVSQLVAQLFQMRVRMKNLMGVMQGGSIPALSNLEEALKTEQKAPPGTARRKRRSESRKQFADSGSTRPGPRGFGAKN

SEQ ID NO: 21 - DNA, 1605 bp, Nicotiana tabacum
  source 1..1605: mol_type = genomic DNA
atgtccgccattgctacttctgctgctctctcttttcctttctctttctgccgttctaccaagacttttgctacaagaaagtgtttcaaagggggatttggagtgtttgcagtgtatgaggaggcaggtgagttaacaaacaagaaaagctcctggttgacactctttgatgtggaagatccaaggtcaaaatttcctcagtctaaaggcaagttcctggatgcaaatcaagctttagaagttgctagatttgatatacaatattgtgattggcgagctcggcaagatgtattaaccataatgctcctgcacgaaaaggttgtggaagtattgaatcctctggcacgtgagtacaaatctattggaaccatgaagaaggaactagcagagttacaaggagcactttctcaggctcataaagaggtacatatatctgaggtgcgggtttctgctgctttagataagctagctcacatggaagcattggttaatgataggctgcttccggagaggagtgcagaagaatcagattgcccgtcttcctccaccggtacgtctacagtatctagagatactgttaaaggcaagcagcctaggagaaccctcaatgtgtcaggtccggtccaagattacagctctcatttgaagaacttttggtatcctgtcgctttttctgctgatgttaaggaagacacaatgacaccaattgattgctttgaggaaccatgggtgatttttcgtgggaaagatggaaaacctggatgtgtccggaacacatgtgcacatagagcctgcccccttcatttgggttcagttaatgagggtcgcatccaatgtccttatcatgggtgggaatattcaacagacggaaaatgtgagaaaatgccatcaactaaatttctgaatgtcaagatcaaagctctgccatgctttgagcaagagggaatgatatggatttggcctggaaatgatcctcctgcagctactcttccttctttactgccaccttctggatttcaaatccatgcagagattgttattgaacttccagtggaacatgggctacttttggacaatctgttggatcttgcacatgctcctttcacccatacgtctacatttgctaaaggatggactgtcccaagctttgtaaaatttttgactcctgcatctggtcttcaaggatattgggatccctatccaatagatatggaatttcgaccaccttgtatagttctatcaaccattggaatctcaaagccaggcaagttggaagggcagagtaccaaagagtgctctacacacctacaccaacttcatgtatgtttacctgcatctaaacagaagacaaggttgttatataggatgtcactggattttgctcccgtgctaaaacacatccctttcatgcaatacgtgtggaggcattttgctgaacaggttttaaacgaagacctacggcttgtgattggtcagcaagagcggatgctcaatggtgctaacatttggaacctgcctgtgtcatacgataagctaggagtgaggtataggatatggagagatgctgtagagagtggagcaaagcaattgccattcagcaaatga

SEQ ID NO: 22 - protein, 534 aa, Nicotiana tabacum
MSAIATSAALSFPFSFCRSTKTFATRKCFKGGFGVFAVYEEAGELTNKKSSWLTLFDVEDPRSKFPQSKGKFLDANQALEVARFDIQYCDWRARQDVLTIMLLHEKVVEVLNPLAREYKSIGTMKKELAELQGALSQAHKEVHISEVRVSAALDKLAHMEALVNDRLLPERSAEESDCPSSSTGTSTVSRDTVKGKQPRRTLNVSGPVQDYSSHLKNFWYPVAFSADVKEDTMTPIDCFEEPWVIFRGKDGKPGCVRNTCAHRACPLHLGSVNEGRIQCPYHGWEYSTDGKCEKMPSTKFLNVKIKALPCFEQEGMIWIWPGNDPPAATLPSLLPPSGFQIHAEIVIELPVEHGLLLDNLLDLAHAPFTHTSTFAKGWTVPSFVKFLTPASGLQGYWDPYPIDMEFRPPCIVLSTIGISKPGKLEGQSTKECSTHLHQLHVCLPASKQKTRLLYRMSLDFAPVLKHIPFMQYVWRHFAEQVLNEDLRLVIGQQERMLNGANIWNLPVSYDKLGVRYRIWRDAVESGAKQLPFSK

SEQ ID NO: 23 - DNA, 1605 bp, Nicotiana tabacum
  source 1..1605: mol_type = genomic DNA
atgtccgccattgctacttctgctgctctctcttttcctttctctttttgccgttctaccaagacttttactacaagaaagtgtttcaaagggggatttggagtgtttgcagtgtatgaggaggcaggtgagttaacaaacaagaaaagctcctggttgacactctttgatgtggaagatccaaggtcaaaatttcctcagtctaaaggcaagttcctggatgcaaatcaagctttagaagttgctagatttgatatgcaatattgtgattggcgagctcggcaagacgtacttacaataatgctcctgcatgaaaaggttgtggaagtattgaatcctctagctcgtgaatataaatctattggaaccatgaagaaggaactcgcggagttacaagaagaactgtctcgggctcacaaagaggtacatatatctgaggtgcgggtttctgctgctttagataagctagctcacatggaagcattggttaatgataggctgcttccggagaggagtacagaagaatcagattccccatcttcctccaccggtacgtctacagtatctagagataatgctaaaagcaagcagcctaggagaaccctcaatgtgtcaggtcccgtccaagattacagctcctatttgaagaacttttggtatcctgtggctttttctgctgatgttaaggaagataccatgacaccaattgattgctttgaggaaccgtgggtgatttttcgtgggaaagatggaaaacctggatgtgtccagaacacatgtgcacatagagcttgcccccttcatttgggttcagtgaatgagggtcgcatacaatgtccttatcacgggtgggaatattcaacagacggaaaatgtgagaaaatgccatcaacaaaatttctgaatgtcaagatcaaagctctgccatgctttgagcaagagggaatgatatggatttggcctggaaacgatcctcctgcagctactcttccttctttgctaccaccttctggatttcaaatccatgcagagattgtcatggaacttccggtggaacatgggctacttttggacaatctgttggatcttgcacatgctcctttcactcatacgtctacatttgctaaaggatggactgtcccaagctttgtaaaatttttgactcctgcgtctggtctgcaaggatattgggatccatatccaatagatatggaatttcgaccgccttgtatggttctgtcaaccattggaatctcaaagccgggcaaattggaagggcagagtatcaaagagtgctctacacaccttcaccaacttcatgtatgtttacctgcatctaaacagaagacaaggttgttatataggatgtcactggattttgctcctgttctaaaacacatccctttcatgcaatacgtgtggaggcattttgctgaacaggttttaaatgaagacctacggcttgtgattggtcagcaagaacggatgctcaatggtgctaacatttggaacctgcctgtgtcatacgataagctaggagtgaggtatagaatatggagagacgctgtagagagtggagcaaagcagttgccgttcagcaaatga

SEQ ID NO: 24 - protein, 534 aa, Nicotiana tabacum
MSAIATSAALSFPFSFCRSTKTFTTRKCFKGGFGVFAVYEEAGELTNKKSSWLTLFDVEDPRSKFPQSKGKFLDANQALEVARFDMQYCDWRARQDVLTIMLLHEKVVEVLNPLAREYKSIGTMKKELAELQEELSRAHKEVHISEVRVSAALDKLAHMEALVNDRLLPERSTEESDSPSSSTGTSTVSRDNAKSKQPRRTLNVSGPVQDYSSYLKNFWYPVAFSADVKEDTMTPIDCFEEPWVIFRGKDGKPGCVQNTCAHRACPLHLGSVNEGRIQCPYHGWEYSTDGKCEKMPSTKFLNVKIKALPCFEQEGMIWIWPGNDPPAATLPSLLPPSGFQIHAEIVMELPVEHGLLLDNLLDLAHAPFTHTSTFAKGWTVPSFVKFLTPASGLQGYWDPYPIDMEFRPPCMVLSTIGISKPGKLEGQSIKECSTHLHQLHVCLPASKQKTRLLYRMSLDFAPVLKHIPFMQYVWRHFAEQVLNEDLRLVIGQQERMLNGANIWNLPVSYDKLGVRYRIWRDAVESGAKQLPFSK

SEQ ID NO: 25 - DNA, 1596 bp, Nicotiana tabacum
  source 1..1596: mol_type = genomic DNA
atgacagccattactactgctctttcttttcctttctctttgtgccgctctactaagtcttatactagaaagtatgtcaaagggagctttggagtgtttgcagtatatggggaggagggtgggatgccagataagaaaagttcctggttgacactctttaatgtggaagatccaaggtctaaagttccacaaattaaaggcaaattcttggatgcaaatcaagctttggaagttgctagatatgatctacaatactgtgattggcgagctcggcaagatgtacttacaatcatgctgctgcatgaaaaggttgtggaagtgttgaaccctctagcacgtgaatacaaatctattggaaccatgaaaaaggaacttgcagagttgcaaggagagctttctcaggcccacaaccaggtacatatatctgaggcccgggtttctgctgctttggataagctagcttacatggaagagttggttaatgataggcttctgcaagagagaagcacggcagaatcagattgctcgtcctcctctgccagtacgtcaacagcattattggatactgttaaaagcaagcaaccccgaagaaccctgagtgtgtcaggtcctgtccaagattacagttcccgtttgaagaacttttggtaccctgttgctttctccgcagatcttaaggatgacaccatgttaccgattgattgctttgagcaaccatgggtgatctttcgcgggaatgatggaaaacctggatgtgtacagaatacgtgtgcacatagagcctgcccccttgatcttggctcagtgaaagagggacgcattcagtgcccttatcacggatgggaatactcaactgatgggaagtgtgagaaaatgccatcaacacgattactgaatgtaaagatcaaagcactgccctgctttgagcaagagggaatgatatggatttggccaggaaatgatccccctgcagctacccttccttctttactaccgccttctggatttcaaatccatgcggagatagtcatggaacttccagtggaacatgggctattattagacaatttattggatcttgcacatgctcctttcacccatacatcaacctttgctaaaggatggagtgtcccaagattggtgaagtttttgactcctgcttctggtctgcaaggatattgggatccttatccaatagatatggaatttagaccaccttgtatggttttatcaaccattggaatctcaaagccaggcaaattggaggggcagagtaccaagcagtgttgtacacaccttcatcaacttcatgtttgcttacctgcatcacgacacaagacacggttattatataggatgtcactggattttgctcccctgctgaaacacatccctttcatgcaatatgtttggagacattttgccgaacaggttttaaatgaagacctacggcttgtgttgggccagcaggatcgcatgctcaatggcgccaatatttggaacttgccagtgtcttacgataagctaggtgtgaggtatagaatatggagagatgctgtagatagtggagcaaagcatctaccattcagcaaataa

SEQ ID NO: 26 - protein, 531 aa, Nicotiana tabacum
MTAITTALSFPFSLCRSTKSYTRKYVKGSFGVFAVYGEEGGMPDKKSSWLTLFNVEDPRSKVPQIKGKFLDANQALEVARYDLQYCDWRARQDVLTIMLLHEKVVEVLNPLAREYKSIGTMKKELAELQGELSQAHNQVHISEARVSAALDKLAYMEELVNDRLLQERSTAESDCSSSSASTSTALLDTVKSKQPRRTLSVSGPVQDYSSRLKNFWYPVAFSADLKDDTMLPIDCFEQPWVIFRGNDGKPGCVQNTCAHRACPLDLGSVKEGRIQCPYHGWEYSTDGKCEKMPSTRLLNVKIKALPCFEQEGMIWIWPGNDPPAATLPSLLPPSGFQIHAEIVMELPVEHGLLLDNLLDLAHAPFTHTSTFAKGWSVPRLVKFLTPASGLQGYWDPYPIDMEFRPPCMVLSTIGISKPGKLEGQSTKQCCTHLHQLHVCLPASRHKTRLLYRMSLDFAPLLKHIPFMQYVWRHFAEQVLNEDLRLVLGQQDRMLNGANIWNLPVSYDKLGVRYRIWRDAVDSGAKHLPFSK

SEQ ID NO: 27 - DNA, 723 bp, synthetic construct
  misc_feature 1..723: note = Description of Artificial Sequence: Synthetic polynucleotide
  source 1..723: mol_type = other DNA
ggatccatggcttctctattatcttctcgtctcccacgtcatctttcctctaataaaccggtactcccaccatcaagctccggttcaaatctccttcacaacttcacatataaaacccggttcgatcaatcccggttcaaatgctcagctggtggaacggggttcttcacgaagttgggtcgtttgctgaaagagaaagcaaagagcgacgtggagaaactgttctcaggattctcaaaaactcgagacaatttagcagttatagatgaactcctcctttactggtaataagatcttcaacacctacaccatttttttaatcactactacccattgcattgaacaaacttccaagttcttcttagcttcagattaagaaagtaccctttcttggctttgttgatgtggtaccattgtccattgtcttgtgtgtttccaccagtaaaggaggagttcatctataactgctaaattgtctcgagtttttgagaatcctgagaacagtttctccacgtcgctctttgctttctctttcagcaaacgacccaacttcgtgaagaaccccgttccaccagctgagcatttgaaccgggattgatcgaaccgggttttatatgtgaagttgtgaaggagatttgaaccggagcttgatggtgggagtaccggtttattagaggaaagatgacgtgggagacgagaagataatagagaagccattctaga

SEQ ID NO: 28 - DNA, 765 bp, synthetic construct
  misc_feature 1..765: note = Description of Artificial Sequence: Synthetic polynucleotide
  source 1..765: mol_type = other DNA
ggatccgggaaaggaatggagtacttgatagagtggaaagacgaacatgccccaacgtgggtcccctctgattacattgctaaagatgttgtggccgagtacgaaactccttggtggaatgcggctaaaaaggccgacgaatccgctcttagggaactcctagaaactgacgacgacagagatgtggacgcagtagatgaggatggacgtacggctttgctctttgtctcgggtctggggtccgagccgtgtgtcaagctgctagctgaagccggcgctgacgtggactatcgcgataggaatggctaataagatcttcaacacctacaccatttttttaatcactactacccattgcattgaacaaacttccaagttcttcttagcttcagattaagaaagtaccctttcttggctttgttgatgtggtaccattgtccattgtcttgtgtgtttccagccattcctatcgcgatagtccacgtcagcgccggcttcagctagcagcttgacacacggctcggaccccagacccgagacaaagagcaaagccgtacgtccatcctcatctactgcgtccacatctctgtcgtcgtcagtttctaggagttccctaagagcggattcgtcggcctttttagccgcattccaccaaggagtttcgtactcggccacaacatctttagcaatgtaatcagaggggacccacgttggggcatgttcgtctttccactctatcaagtactccattcctttccctctaga

SEQ ID NO: 29 - DNA, 747 bp, synthetic construct
  misc_feature 1..747: note = Description of Artificial Sequence: Synthetic polynucleotide
  source 1..747: mol_type = other DNA
ggatccatggaagccactgcttctttctcctcaactatgtcttcccaccatttctttccactttccaaagccaccctctcaacttctaaacttccattttctgggactggttcaactcattctctttcattttcttcaagaaactcattcactagggatacatggagggtgatcaattcaaggaatgtggttatttcaagaagagaaatgcgtggagttattagagctgagatgtttggacagctcactagtggacttgaatcagcttggaataagctcaaaggagaagaggttttgtaataagatcttcaacacctacaccatttttttaatcactactacccattgcattgaacaaacttccaagttcttcttagcttcagattaagaaagtaccctttcttggctttgttgatgtggtaccattgtccattgtcttgtgtgtttccacaaaacctcttctcctttgagcttattccaagctgattcaagtccactagtgagctgtccaaacatctcagctctaataactccacgcatttctcttcttgaaataaccacattccttgaattgatcaccctccatgtatccctagtgaatgagtttcttgaagaaaatgaaagagaatgagttgaaccagtcccagaaaatggaagtttagaagttgagagggtggctttggaaagtggaaagaaatggtgggaagacatagttgaggagaaagaagcagtggcttccattctaga

SEQ ID NO: 30 - DNA, 987 bp, synthetic construct
  misc_feature 1..987: note = Description of Artificial Sequence: Synthetic polynucleotide
  source 1..987: mol_type = other DNA
ggatccgaggcaggtgagttaacaaacaagaaaagctcctggttgacactctttgatgtggaagatccaaggtcaaaatttcctcagtctaaaggcaagttcctggatgcaaatcaagctttagaagttgctagatttgatatacaatattgtgattggcgagctcggcaagatgtattaaccataatgctcctgcacgaaaaggttgtggaagtattgaatcctctggcacgtgagtacaaatctattggaaccatgaaaaaggaacttgcagagttgcaaggagagctttctcaggcccacaaccaggtacatatatctgaggcccgggtttctgctgctttggataagctagcttacatggaagagttggttaatgataggcttctgcaagagagaagcacggcagaatcagattaataagatcttcaacacctacaccatttttttaatcactactacccattgcattgaacaaacttccaagttcttcttagcttcagattaagaaagtaccctttcttggctttgttgatgtggtaccattgtccattgtcttgtgtgtttccaatctgattctgccgtgcttctctcttgcagaagcctatcattaaccaactcttccatgtaagctagcttatccaaagcagcagaaacccgggcctcagatatatgtacctggttgtgggcctgagaaagctctccttgcaactctgcaagttcctttttcatggttccaatagatttgtactcacgtgccagaggattcaatacttccacaaccttttcgtgcaggagcattatggttaatacatcttgccgagctcgccaatcacaatattgtatatcaaatctagcaacttctaaagcttgatttgcatccaggaacttgcctttagactgaggaaattttgaccttggatcttccacatcaaagagtgtcaaccaggagcttttcttgtttgttaactcacctgcctctctaga

SEQ ID NO: 31 - DNA, 3155 bp, synthetic construct
  misc_feature 1..3155: note = Description of Artificial Sequence: Synthetic polynucleotide
  misc_feature 1..480: note = MISC_FEATURE - pCSVMV Promoter
  misc_feature 523..551: note = MISC_FEATURE - Cloning region
  misc_feature 601..840: note = MISC_FEATURE - NOS terminator
  misc_feature 841..1980: note = MISC_FEATURE - ACTII Promoter
  misc_feature 2101..2820: note = MISC_FEATURE - NPT II Kan Resistance
  misc_feature 2941..3120: note = MISC_FEATURE - NOS Terminator
  source 1..3155: mol_type = other DNA
aagcttccagaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtggatcctgcaggctagcgtgcactctagactcgacgaactgacgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactatgataaattatcgcgcgcggtgtcatctatgttactagatcgggaattcctcgagcaactatttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcagctcataaaaggtaccaaacaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttttgagcgggactctggcgatcgccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactagt

SEQ ID NO: 32 - protein, 20 aa, Nicotiana tabacum
NGGGKTTSLGKLANRLKKEG

SEQ ID NO: 33 - protein, 23 aa, Nicotiana tabacum
RGGCVVSVVDELGIPVKFVGVGE

SEQ ID NO: 34 - protein, 20 aa, Nicotiana tabacum
KRGKGENVEYLVKWKDGEDN

SEQ ID NO: 35 - protein, 22 aa, Nicotiana tabacum
RTALLFVSGLGSEPCVKLLAEA

SEQ ID NO: 36 - protein, 20 aa, Nicotiana tabacum
RRSESRKQFADSGSTRPGPR

SEQ ID NO: 37 - protein, 18 aa, Nicotiana tabacum
LKEVKRVLNPTEVLLVVD

SEQ ID NO: 38 - DNA, 27 bp, synthetic construct
  misc_feature 1..27: note = Description of Artificial Sequence: Synthetic primer
  source 1..27: mol_type = other DNA
atggcttctctattatcttctcgtctc

SEQ ID NO: 39 - DNA, 25 bp, synthetic construct
  misc_feature 1..25: note = Description of Artificial Sequence: Synthetic primer
  source 1..25: mol_type = other DNA
gttcaaatgctcagctggtggaacg

SEQ ID NO: 40 - DNA, 25 bp, synthetic construct
  misc_feature 1..25: note = Description of Artificial Sequence: Synthetic primer
  source 1..25: mol_type = other DNA
cgtcaattcctctctctcccgcctc

SEQ ID NO: 41 - DNA, 25 bp, synthetic construct
  misc_feature 1..25: note = Description of Artificial Sequence: Synthetic primer
  source 1..25: mol_type = other DNA
cttcagaaccagcagcaacaagcag

SEQ ID NO: 42 - DNA, 26 bp, synthetic construct
  misc_feature 1..26: note = Description of Artificial Sequence: Synthetic primer
  source 1..26: mol_type = other DNA
atggaagccactgcttctttctcctc

SEQ ID NO: 43 - DNA, 28 bp, synthetic construct
  misc_feature 1..28: note = Description of Artificial Sequence: Synthetic primer
  source 1..28: mol_type = other DNA
ctcattcactagggatacatggagggtg

SEQ ID NO: 44 - protein, 6 aa, synthetic construct
  REGION 1..6: note = Description of Artificial Sequence: Synthetic 6xHis tag
HHHHHH

SEQ ID NO: 45 - DNA, 285 bp, synthetic construct
  misc_feature 1..285: note = Description of Artificial Sequence: Synthetic polynucleotide
  source 1..285: mol_type = other DNA
ggatccatggcttctctattatcttctcgtctcccacgtcatctttcctctaataaaccggtactcccaccatcaagctccggttcaaatctccttcacaacttcacatataaaacccggttcgatcaatcccggttcaaatgctcagctggtggaacggggttcttcacgaagttgggtcgtttgctgaaagagaaagcaaagagcgacgtggagaaactgttctcaggattctcaaaaactcgagacaatttagcagttatagatgaactcctcctttactgg

SEQ ID NO: 46 - DNA, 153 bp, synthetic construct
  misc_feature 1..153: note = Description of Artificial Sequence: Synthetic polynucleotide
  source 1..153: mol_type = other DNA
taataagatcttcaacacctacaccatttttttaatcactactacccattgcattgaacaaacttccaagttcttcttagcttcagattaagaaagtaccctttcttggctttgttgatgtggtaccattgtccattgtcttgtgtgtttcca

SEQ ID NO: 47 - DNA, 285 bp, synthetic construct
  misc_feature 1..285: note = Description of Artificial Sequence: Synthetic polynucleotide
  source 1..285: mol_type = other DNA
ccagtaaaggaggagttcatctataactgctaaattgtctcgagtttttgagaatcctgagaacagtttctccacgtcgctctttgctttctctttcagcaaacgacccaacttcgtgaagaaccccgttccaccagctgagcatttgaaccgggattgatcgaaccgggttttatatgtgaagttgtgaaggagatttgaaccggagcttgatggtgggagtaccggtttattagaggaaagatgacgtgggagacgagaagataatagagaagccattctaga

SEQ ID NO: 48 - DNA, 306 bp, synthetic construct
  misc_feature 1..306: note = Description of Artificial Sequence: Synthetic polynucleotide
  source 1..306: mol_type = other DNA
ggatccgggaaaggaatggagtacttgatagagtggaaagacgaacatgccccaacgtgggtcccctctgattacattgctaaagatgttgtggccgagtacgaaactccttggtggaatgcggctaaaaaggccgacgaatccgctcttagggaactcctagaaactgacgacgacagagatgtggacgcagtagatgaggatggacgtacggctttgctctttgtctcgggtctggggtccgagccgtgtgtcaagctgctagctgaagccggcgctgacgtggactatcgcgataggaatggc

SEQ ID NO: 49 - DNA, 153 bp, synthetic construct; identical to SEQ ID NO: 46
  misc_feature 1..153: note = Description of Artificial Sequence: Synthetic polynucleotide
  source 1..153: mol_type = other DNA

SEQ ID NO: 50 - DNA, 306 bp, synthetic construct
  misc_feature 1..306: note = Description of Artificial Sequence: Synthetic polynucleotide
  source 1..306: mol_type = other DNA
gccattcctatcgcgatagtccacgtcagcgccggcttcagctagcagcttgacacacggctcggaccccagacccgagacaaagagcaaagccgtacgtccatcctcatctactgcgtccacatctctgtcgtcgtcagtttctaggagttccctaagagcggattcgtcggcctttttagccgcattccaccaaggagtttcgtactcggccacaacatctttagcaatgtaatcagaggggacccacgttggggcatgttcgtctttccactctatcaagtactccattcctttccctctaga

SEQ ID NO: 51 - DNA, 297 bp, synthetic construct
  misc_feature 1..297: note = Description of Artificial Sequence: Synthetic polynucleotide
  source 1..297: mol_type = other DNA
ggatccatggaagccactgcttctttctcctcaactatgtcttcccaccatttctttccactttccaaagccaccctctcaacttctaaacttccattttctgggactggttcaactcattctctttcattttcttcaagaaactcattcactagggatacatggagggtgatcaattcaaggaatgtggttatttcaagaagagaaatgcgtggagttattagagctgagatgtttggacagctcactagtggacttgaatcagcttggaataagctcaaaggagaagaggttttg

SEQ ID NO: 52 - DNA, 153 bp, synthetic construct; identical to SEQ ID NO: 46
  misc_feature 1..153: note = Description of Artificial Sequence: Synthetic polynucleotide
  source 1..153: mol_type = other DNA

SEQ ID NO: 53 - DNA, 297 bp, synthetic construct
  misc_feature 1..297: note = Description of Artificial Sequence: Synthetic polynucleotide
  source 1..297: mol_type = other DNA
caaaacctcttctcctttgagcttattccaagctgattcaagtccactagtgagctgtccaaacatctcagctctaataactccacgcatttctcttcttgaaataaccacattccttgaattgatcaccctccatgtatccctagtgaatgagtttcttgaagaaaatgaaagagaatgagttgaaccagtcccagaaaatggaagtttagaagttgagagggtggctttggaaagtggaaagaaatggtgggaagacatagttgaggagaaagaagcagtggcttccattctaga

SEQ ID NO: 54 - DNA, 417 bp, synthetic construct
  misc_feature 1..417: note = Description of Artificial Sequence: Synthetic polynucleotide
  source 1..417: mol_type = other DNA
ggatccgaggcaggtgagttaacaaacaagaaaagctcctggttgacactctttgatgtggaagatccaaggtcaaaatttcctcagtctaaaggcaagttcctggatgcaaatcaagctttagaagttgctagatttgatatacaatattgtgattggcgagctcggcaagatgtattaaccataatgctcctgcacgaaaaggttgtggaagtattgaatcctctggcacgtgagtacaaatctattggaaccatgaaaaaggaacttgcagagttgcaaggagagctttctcaggcccacaaccaggtacatatatctgaggcccgggtttctgctgctttggataagctagcttacatggaagagttggttaatgataggcttctgcaagagagaagcacggcagaatcagat

SEQ ID NO: 55 - DNA, 153 bp, synthetic construct; identical to SEQ ID NO: 46
  misc_feature 1..153: note = Description of Artificial Sequence: Synthetic polynucleotide
  source 1..153: mol_type = other DNA

SEQ ID NO: 56 - DNA, 417 bp, synthetic construct
  misc_feature 1..417: note = Description of Artificial Sequence: Synthetic polynucleotide
  source 1..417: mol_type = other DNA
atctgattctgccgtgcttctctcttgcagaagcctatcattaaccaactcttccatgtaagctagcttatccaaagcagcagaaacccgggcctcagatatatgtacctggttgtgggcctgagaaagctctccttgcaactctgcaagttcctttttcatggttccaatagatttgtactcacgtgccagaggattcaatacttccacaaccttttcgtgcaggagcattatggttaatacatcttgccgagctcgccaatcacaatattgtatatcaaatctagcaacttctaaagcttgatttgcatccaggaacttgcctttagactgaggaaattttgaccttggatcttccacatcaaagagtgtcaaccaggagcttttcttgtttgttaactcacctgcctctctaga

(19) United States
(12) Patent Application Publication
(10) Pub. No.: US 2024/0150783 A1
(43) Pub. Date: May 9, 2024

(54) TOBACCO PLANTS EXHIBITING ALTERED PHOTOSYNTHESIS AND METHODS OF MAKING AND USING

(71) Applicant: ALTRIA CLIENT SERVICES LLC, Richmond, VA (US)

(72) Inventors: Yanxin SHEN, Glen Allen, VA (US); Dongmei XU, Glen Allen, VA (US)

(73) Assignee: ALTRIA CLIENT SERVICES LLC, Richmond, VA (US)

(21) Appl. No.: 18/505,449

(22) Filed: Nov. 9, 2023

Related U.S. Application Data
(63) Continuation of application No. 17/518,303, filed on Nov. 3, 2021, now Pat. No. 11,845,946.
Application No. 17/518,303 is a continuation of application No. 16/579,271, filed on Sep. 23, 2019, now Pat. No. 11,174,490.
Application No. 16/579,271 is a continuation of application No. 15/486,841, filed on Apr. 13, 2017, now Pat. No. 10,457,953.
(60) Provisional application No. 62/322,001, filed on Apr. 13, 2016.

(51) Int. Cl. C12N 15/82; A01H 1/06; A01H 5/12; A24B 13/00; C07K 14/415; C12N 9/02

(52) U.S. Cl. CPC C12N 15/8261; A01H 1/06; A01H 5/12; A24B 13/00; C07K 14/415; C12N 9/0073; C12N 15/8218; C12N 15/825; C12N 15/8251; C12N 15/8269; C12Y 114/13122; Y02A 40/146

(57) ABSTRACT

This disclosure provides tobacco plants that exhibit altered photosynthesis as well as methods of making and using such plants.

### CROSS REFERENCE TO RELATED APPLICATIONS

This application is a continuation of U.S. patent application Ser. No. 17/518,303, filed Nov. 3, 2021, which is a continuation of U.S. patent application Ser. No. 16/579,271, filed Sep. 23, 2019, now U.S. Pat. No. 11,174,490, issued Nov. 16, 2021, which is a continuation of U.S. patent application Ser. No. 15/486,841, filed Apr. 13, 2017, now U.S. Pat. No. 10,457,953, issued Oct. 29, 2019, which in turn claims the benefit of priority under 35 U.S.C. § 119(e) to U.S. application Ser. No. 62/322,001 filed Apr. 13, 2016.

### INCORPORATION OF THE SEQUENCE LISTING

The Sequence Listing is hereby incorporated by reference in its entirety, including the file named “P34456US05_ST26,” which is 90,185 bytes in size and was created on Nov. 7, 2023, which is herein incorporated by reference in its entirety.

### TECHNICAL FIELD

This disclosure generally relates to plants that exhibit altered photosynthesis.

### BACKGROUND

During photosynthesis, at low sunlight intensities, all absorbed photons are utilized efficiently to drive electrons in the electron-transport chain. As the level of irradiance increases further, photosynthesis becomes saturated and reaches a plateau due to the fact that the carbon reactions cannot keep up with the linear increase in light absorption. Plant lines with a wild type light-harvesting antenna system reach this light intensity for saturation at lower levels of irradiance than their mutant counterparts. The sunlight harvested by the chlorophyll antenna exceeds the maximal operational capacity of the electron-transport chain and of the carbon reactions of photosynthesis, rendering the excess absorbed photons useless.

Under bright sunlight conditions (2500 μmol photons m−2 s−1), wild type lines with their fully developed light-harvesting antenna utilize photons inefficiently; only about 20% of the incoming sunlight energy is converted into useful photosynthesis, while excess absorbed energy is dissipated by the non-photochemical quenching (NPQ) process.

### SUMMARY

Tobacco plants that exhibit altered photosynthesis are provided herein, as well as methods of making and using such plants.

In one aspect, a method of making a Nicotiana tabacum plant is provided. Such a method typically includes inducing mutagenesis in N. tabacum cells to produce mutagenized N. tabacum cells; obtaining one or more N. tabacum plants from the mutagenized N. tabacum cells; and identifying at least one of the N. tabacum plants that comprises a mutated TLA or CAO sequence. Representative TLA or CAO sequences have at least 95% sequence identity to a sequence shown in SEQ ID NO: 11, 13, 15, 17, 19, 21, 23 or 25.

Such a method further can include identifying at least one of the N. tabacum plants that exhibits reduced amount of TLA or CAO mRNA; reduced amount of TLA or CAO polypeptide; reduced activity of a TLA or CAO polypeptide; reduced concentration of thylakoid membranes in the photosystems; reduced amount of total chlorophyll; increased ratio of chlorophyll a to chlorophyll b; and/or increased biomass relative to a N. tabacum plant lacking a mutated TLA or CAO sequence.

In some embodiments, leaf from the mutant N. tabacum plant exhibits comparable or better quality than leaf from the plant lacking a mutated TLA or CAO sequence. In some embodiments, the N. tabacum plant is a Burley type, a dark type, a flue-cured type, or an Oriental type.

In another aspect, a variety of Nicotiana tabacum is provided. Such a variety typically includes plants having a mutation in an endogenous nucleic acid, where the wild type endogenous nucleic acid encodes the TLA or CAO sequence shown in SEQ ID NO:12, 14, 16, 18, 20, 22, 24 or 26. Typically, leaf from the mutant plants exhibits reduced amount of TLA or CAO mRNA; reduced amount of TLA or CAO polypeptide; reduced activity of a TLA or CAO polypeptide; reduced concentration of thylakoid membranes in the photosystems; reduced amount of total chlorophyll; increased ratio of chlorophyll a to chlorophyll b; and/or increased biomass relative to leaf from a plant lacking the mutation. In some embodiments, leaf from the mutant N. tabacum plant exhibits comparable or better quality than leaf from the plant lacking a mutated TLA sequence.

In another aspect, cured leaf from one of the N. tabacum varieties described herein is provided. In still another aspect, a tobacco product that includes such cured leaf is provided. Representative tobacco products include, without limitation, cigarettes, smokeless tobacco products, tobacco-derived nicotine products, cigarillos, non-ventilated recess filter cigarettes, vented recess filter cigarettes, cigars, snuff, electronic cigarettes, e-vapor products, pipe tobacco, cigar tobacco, cigarette tobacco, chewing tobacco, leaf tobacco, shredded tobacco, and cut tobacco.

In still another aspect, a RNA nucleic acid molecule is provided. Such a RNA nucleic acid molecule typically includes a first nucleic acid between 15 and 500 nucleotides in length and a second nucleic acid between 15 and 500 nucleotides in length, where the first nucleic acid has a region of complementarity to the second nucleic acid, and where the first nucleic acid comprises at least 15 contiguous nucleotides of the sequence shown in SEQ ID NO: 11, 13, 15, 17, 19, 21, 23 or 25. In some embodiments, the RNA nucleic acid molecule further includes a spacer nucleic acid between the first nucleic acid and the second nucleic acid.

In one aspect, a method of making a Nicotiana tabacum plant is provided. Such a method typically includes transforming N. tabacum cells with the nucleic acid molecule of claim 12 to produce transgenic N. tabacum cells; regenerating transgenic N. tabacum plants from the transgenic N. tabacum cells; and selecting at least one transgenic N. tabacum plant that comprises the nucleic acid molecule or the construct. In some embodiments, such a method further includes identifying at least one transgenic N. tabacum plant having reduced amount of TLA or CAO mRNA; reduced amount of TLA or CAO polypeptide; reduced activity of a TLA or CAO polypeptide; reduced concentration of thylakoid membranes in the photosystems; reduced amount of total chlorophyll; increased ratio of chlorophyll a to chlorophyll b; and/or increased biomass relative to a N. tabacum plant not transformed with the nucleic acid molecule. In some embodiments, leaf from the selected transgenic N. tabacum plant exhibits comparable or better quality than leaf from the non-transformed N. tabacum plant.

In another aspect, a transgenic Nicotiana tabacum plant is provided that includes a vector, where the vector includes a RNA nucleic acid molecule having a length of 15 to 500 nucleotides and has at least 95% sequence identity to a TLA or CAO nucleic acid shown in SEQ ID NO: 11, 13, 15, 17, 19, 21, 23 or 25. In some embodiments, leaf from the plant exhibits comparable or better quality than leaf from a N. tabacum plant lacking the nucleic acid molecule.

In one aspect, cured leaf from such transgenic N. tabacum plants is provided. In one aspect, tobacco products that include such cured leaf are provided. Representative tobacco products include, without limitation, smokeless tobacco products, tobacco-derived nicotine products, cigarillos, non-ventilated recess filter cigarettes, vented recess filter cigarettes, cigars, snuff, pipe tobacco, cigar tobacco, cigarette tobacco, chewing tobacco, leaf tobacco, shredded tobacco, and cut tobacco.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### BRIEF DESCRIPTION OF THE DRAWINGS

FIG. 1A is a photograph of a wild type tobacco plant (left) and a T0 tobacco plant transgenic for a RNAi nucleic acid molecule directed toward TLA2 (TLA2i-1; right).

FIG. 1B is a photograph of a T1 tobacco plant transgenic for a RNAi nucleic acid molecule directed toward TLA2 (TLA2i-1).

FIG. 2A is a photograph of T0 tobacco plants transgenic for a RNAi nucleic acid molecule directed toward TLA3 (the three plants on the left, from left to right: TLA3i-5, TLA3i-2, and TLA3i-3) and a wild type tobacco plant (far right).

FIG. 2B is a photograph of a T1 tobacco plant transgenic for a nucleic acid molecule directed toward TLA3 (TLA3i-3).

FIG. 2C is a photograph of a T1 tobacco plant transgenic for a nucleic acid molecule directed toward TLA3 (TLA3i1-1).

FIG. 3 is a photograph of a wild type tobacco plant (left) and a T0 tobacco plant transgenic for a RNAi nucleic acid molecule directed toward TLA4 (TLA4; right).

FIG. 4A is a graph showing the amount of TLA2 mRNA in T0 tobacco plants transgenic for RNAi nucleic acid molecules directed toward TLA2.

FIG. 4B is a graph showing the amount of TLA3 mRNA in T0 tobacco plants transgenic for RNAi nucleic acid molecules directed toward TLA3.

FIG. 5A is a photograph of a T0 generation tobacco plant transgenic for CAOi-1.

FIG. 5B is a photograph of a T0 generation tobacco plant transgenic for CAOi-2.

### DETAILED DESCRIPTION

Photosynthetic organisms (e.g., green plants, algae, and many bacteria) contain reaction centers, which is a complex of proteins, pigments and co-factors that perform the photosynthetic conversion of light to energy via a multitude of electron transfer steps. Despite the evolutionary distances between such photosynthetic organisms, the reaction centers possess remarkable homology. In contrast, the light-harvesting complexes in the various photosynthetic organisms differ. The current methods used to measure light absorption and utilization in plants and microalgae are described in Melis and Thielen (1980, Biochim. Biophys. Acta, 589:275-86) and Melis (1989, Philos. Trans. R. Soc. Lond. B, 323:397-409).

During the process of photosynthesis, the light-harvesting complex, which typically surrounds the reaction center, absorbs the light (e.g., sunlight). In plants, the light energy is absorbed by the light-harvesting antenna complex and is transferred to two chlorophyll a molecules, which are embedded in the reaction center. As described herein, light-harvesting antenna complex size can be inhibited or reduced in tobacco using, for example, mutagenesis or RNAi, to diminish over-absorption of sunlight at the higher canopy. Diminishing over-absorption of sunlight in the higher canopy of the plant can minimize wasteful dissipation of energy, while, at the same time, allowing for a far greater transmittance of sunlight deeper into the lower canopy by eliminating unwanted shading, particularly under high density growth conditions.

Tobacco genes and the encoded proteins were screened to identify those involved in harvesting light; those sequences identified in the screen were evaluated further to identify their mode of action. The sequences identified herein can be inhibited (e.g., by RNA interference and/or mutation) to result in smaller light-harvesting chlorophyll antenna size, which ultimately results in a plant that exhibits substantially improved photosynthetic efficiency. A number of tobacco sequences (e.g., truncated light-harvesting antenna (TLA) 2, TLA3, TLA4 and CAO (Chlorophyllide a oxygenase)) as well as corresponding homologues from Chlamydomonas and/or Arabidopsis were obtained.

Four TLA-related genes, TLA2 and TLA2 Homo (encoding the CpFTSY protein), TLA3 and TLA3 Homo (encoding theCpSRP43 protein), and TLA4 (encoding the CpSRP54 protein) were obtained from Nicotiana tabacum, as well as three CAO genes (CAO-2, COA-3 and CAO-4). Based on sequence alignment, CAO-2 appears to have originated from Nicotiana tometosiformis, while both CAO-3 and COA-4 originated from Nicotiana sylvestris.

As described in more detail below, the expression of one or more of the sequences described herein can be inhibited or reduced using, for example, mutagenesis or inhibitory RNA (RNAi). The resulting plants can be evaluated for total chlorophyll, as well as the ratio of chlorophyll a:chlorophyll b and/or the photosynthetic apparatus size in Photosystem I (PSI) and/or Photosystem II (PSII). Sequences that, when their expression is knocked down or completely eliminated, result in a higher ratio of chlorophyll a:b and reduced antenna size in PSI and/or PSII systems were desired, as it is these sequences that will substantially improve photosynthetic efficiency.

As described herein, modification of TLA sequences and CAO sequences in tobacco results in smaller light-harvesting chlorophyll antenna complex size by reducing antenna number and a substantially improved photosynthetic efficiency. The modified tobacco lines further exhibit enhanced productivity (e.g., increased biomass).

Specifically, for example, knocking down TLA2 resulted in plants that grow slower than wild type plants, plants that have a ratio of Chlorophyll a:Chlorophyll b similar to wild type, plants that have an antenna size in PSI that is similar to wild type, plants that have an antenna size in PSII that is reduced compared to wild type plants, and plants that have a lighter leaf color than wild type plants due to the reduction of total chlorophyll content.

In addition, knocking down TLA3 resulted in plants that grow at a similar rate to wild type plants, plants that have a ratio of Chlorophyll a:Chlorophyll b that is increased relative to wild type plants, plants that have an antenna size in both PSI and PSII that is reduced compared to wild type plants, plants in which the amount of total chlorophyll increased from low to normal levels during maturation, relative to wild type plants.

Further, knocking down TLA4 resulted in plants in which the amount of total chlorophyll was reduced, but both the ratio of chlorophyll a:chlorophyll b and the antenna size in both PSI and PSII were unchanged.

### Light Harvesting Antenna Nucleic Acids and Polypeptides

Nucleic acids encoding TLA2, TLA2-homo, TLA3, TLA3-homo and TLA4 from N. tabacum are shown in SEQ ID NOs: 11, 13, 15, 17, and 19, respectively, and nucleic acids encoding CAO2, CAO3 and CAO4 from N. tabacum are shown in SEQ ID NOs: 21, 23, and 25, respectively. Unless otherwise specified, nucleic acids referred to herein can refer to DNA and RNA, and also can refer to nucleic acids that contain one or more nucleotide analogs or backbone modifications. Nucleic acids can be single stranded or double stranded, and linear or circular, both of which usually depend upon the intended use.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule, discussed in more detail below. In addition, an isolated nucleic acid molecule can include an engineered nucleic acid molecule such as a recombinant or a synthetic nucleic acid molecule.

The sequence of the TLA2, TLA2-homo, TLA3, TLA3-homo and TLA4 polypeptides from N. tabacum are shown in SEQ ID NOs: 12, 14, 16, 18, and 20, respectively, and the sequences of the CAO2, CAO3 and CAO4 polypeptides from N. tabacum are shown in SEQ ID NOs: 22, 24, and 26, respectively. As used herein, a “purified” polypeptide is a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the polypeptides and naturally occurring molecules with which it is naturally associated. Since a polypeptide that is chemically synthesized is, by nature, separated from the components that naturally accompany it, a synthetic polypeptide is “purified.”

Nucleic acids can be isolated using techniques well known in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Nucleic acids can be detected using any number of amplification techniques (see, e.g., PCR Primer: A Laboratory Manual, 1995, Dieffenbach & Dveksler, Eds., Cold Spring Harbor Laboratory Press, Cold Spring Harbor, NY; and U.S. Pat. Nos. 4,683,195; 4,683,202; 4,800,159; and 4,965,188) with an appropriate pair of oligonucleotides (e.g., primers). A number of modifications to the original PCR have been developed and can be used to detect a nucleic acid. Nucleic acids also can be detected using hybridization.

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is oftentimes accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

A construct, sometimes referred to as a vector, containing a nucleic acid (e.g., a coding sequence or a RNAi nucleic acid molecule) is provided. Constructs, including expression constructs (or expression vectors), are commercially available or can be produced by recombinant DNA techniques routine in the art. A construct containing a nucleic acid can have expression elements operably linked to such a nucleic acid, and further can include sequences such as those encoding a selectable marker (e.g., an antibiotic resistance gene). A construct can encode a chimeric or fusion polypeptide (i.e., a first polypeptide operatively linked to a second polypeptide). Representative first (or second) polypeptides are those that can be used in purification of the other (i.e., second (or first), respectively) polypeptide including, without limitation, 6xHis (SEQ ID NO:44) tag or glutathione S-transferase (GST).

Expression elements include nucleic acid sequences that direct and regulate expression of nucleic acid coding sequences. One example of an expression element is a promoter sequence. Expression elements also can include introns, enhancer sequences, response elements, or inducible elements that modulate expression of a nucleic acid. Expression elements can be of bacterial, yeast, insect, mammalian, or viral origin, and vectors can contain a combination of elements from different origins. As used herein, operably linked means that a promoter or other expression element(s) are positioned in a vector relative to a nucleic acid in such a way as to direct or regulate expression of the nucleic acid (e.g., in-frame).

Constructs as described herein can be introduced into a host cell. Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer. As used herein, “host cell” refers to the particular cell into which the nucleic acid is introduced and also includes the progeny or potential progeny of such a cell. A host cell can be any prokaryotic or eukaryotic cell. For example, nucleic acids can be introduced into bacterial cells such as E. coli, or into insect cells, yeast or mammalian cells (such as Chinese hamster ovary cells (CHO) or COS cells). Other suitable host cells are known to those skilled in the art.

### RNA Interfering Nucleic Acids and Constructs Containing Same

RNA interference (RNAi), also called post-transcriptional gene silencing (PTGS), is a biological process in which RNA molecules inhibit gene expression, typically by causing the destruction of specific mRNA molecules. Without being bound by theory, it appears that, in the presence of an antisense RNA molecule that is complementary to an expressed message (i.e., a mRNA), the two strands anneal to generate long double-stranded RNA (dsRNA), which is digested into short (<30 nucleotide) RNA duplexes, known as small interfering RNAs (siRNAs), by an enzyme known as Dicer. A complex of proteins known as the RNA Induced Silencing Complex (RISC) then unwinds siRNAs, and uses one strand to identify and thereby anneal to other copies of the original mRNA. RISC cleaves the mRNA within the complementary sequence, leaving the mRNA susceptible to further degradation by exonucleases, which effectively silences expression of the encoding gene.

Several methods have been developed that take advantage of the endogenous machinery to suppress the expression of a specific target gene and a number of companies offer RNAi design and synthesis services (e.g., Life Technologies, Applied Biosystems). In transgenic plants, the use of RNAi can involve the introduction of long dsRNA (e.g., greater than 50 bps) or siRNAs (e.g., 12 to 23 bps) that have complementarity to the target gene, both of which are processed by the endogenous machinery. Alternatively, the use of RNAi can involve the introduction of a small hairpin RNA (shRNA); shRNA is a nucleic acid that includes the sequence of the two desired siRNA strands, sense and antisense, on a single strand, connected by a “loop” or “spacer” nucleic acid. When the shRNA is transcribed, the two complementary portions anneal intra-molecularly to form a “hairpin,” which is recognized and processed by the endogenous machinery.

A RNAi nucleic acid molecule as described herein is complementary to at least a portion of a target mRNA (e.g., a TLA mRNA, a CAO mRNA), and typically is referred to as an “antisense strand”. Typically, the antisense strand includes at least 15 contiguous nucleotides of the DNA sequence (e.g., the nucleic acid sequence shown in SEQ ID NO:11, 13, 15, 17, 19, 21, 23, or 25); it would be appreciated that the antisense strand has the “RNA equivalent” sequence of the DNA (e.g., uracils instead of thymines; ribose sugars instead of deoxyribose sugars).

A RNAi nucleic acid molecule can be, for example, 15 to 500 nucleotides in length (e.g., 15 to 50, 15 to 45, 15 to 30, 16 to 47, 16 to 38, 16 to 29, 17 to 53, 17 to 44, 17 to 38, 18 to 36, 19 to 49, 20 to 60, 20 to 40, 25 to 75, 25 to 100, 28 to 85, 30 to 90, 15 to 100, 15 to 300, 15 to 450, 16 to 70, 16 to 150, 16 to 275, 17 to 74, 17 to 162, 17 to 305, 18 to 60, 18 to 75, 18 to 250, 18 to 400, 20 to 35, 20 to 60, 20 to 80, 20 to 175, 20 to 225, 20 to 325, 20 to 400, 20 to 475, 25 to 45, 25 to 65, 25 to 100, 25 to 200, 25 to 250, 25 to 300, 25 to 350, 25 to 400, 25 to 450, 30 to 280, 35 to 250, 200 to 500, 200 to 400, 250 to 450, 250 to 350, or 300 to 400 nucleotides in length).

In some embodiments, the antisense strand (e.g., a first nucleic acid) can be accompanied by a “sense strand” (e.g., a second nucleic acid), which is complementary to the antisense strand. In the latter case, each nucleic acid (e.g., each of the sense and antisense strands) can be between 15 and 500 nucleotides in length (e.g., between 15 to 50, 15 to 45, 15 to 30, 16 to 47, 16 to 38, 16 to 29, 17 to 53, 17 to 44, 17 to 38, 18 to 36, 19 to 49, 20 to 60, 20 to 40, 25 to 75, 25 to 100, 28 to 85, 30 to 90, 15 to 100, 15 to 300, 15 to 450, 16 to 70, 16 to 150, 16 to 275, 17 to 74, 17 to 162, 17 to 305, 18 to 60, 18 to 75, 18 to 250, 18 to 400, 20 to 35, 20 to 60, 20 to 80, 20 to 175, 20 to 225, 20 to 325, 20 to 400, 20 to 475, 25 to 45, 25 to 65, 25 to 100, 25 to 200, 25 to 250, 25 to 300, 25 to 350, 25 to 400, 25 to 450, 30 to 25 280, 35 to 250, 200 to 500, 200 to 400, 250 to 450, 250 to 350, or 300 to 400 nucleotides in length).

In some embodiments, a spacer nucleic acid, sometimes referred to as a loop nucleic acid, can be positioned between the sense strand and the antisense strand. In some embodiments, the spacer nucleic acid can be an intron (see, for example, Wesley et al., 2001, The Plant J., 27:581-90). In some embodiments, although not required, the intron can be functional (i.e., in sense orientation; i.e., spliceable) (see, for example, Smith et al., 2000, Nature, 407:319-20). A spacer nucleic acid can be between 20 nucleotides and 1000 nucleotides in length (e.g., 25-800, 25-600, 25-400, 50-750, 50-500, 50-250, 100-700, 100-500, 100-300, 250-700, 300-600, 400-700, 500-800, 600-850, or 700-1000 nucleotides in length).

In some embodiments, a construct can be produced by operably linking a promoter that is operable in plant cells; a DNA region, that, when transcribed, produces an RNA molecule capable of forming a hairpin structure; and a DNA region involved in transcription termination and polyadenylation. It would be appreciated that the hairpin structure has two annealing RNA sequences, where one of the annealing RNA sequences of the hairpin RNA structure includes a sense sequence identical to at least 20 consecutive nucleotides of a TLA or CAO nucleotide sequence, and where the second of the annealing RNA sequences includes an antisense sequence that is identical to at least 20 consecutive nucleotides of the complement of the TLA or CAO nucleotide sequence. In addition, as indicated herein, the DNA region can include an intron (e.g., a functional intron). When present, the intron generally is located between the two annealing RNA sequences in sense orientation such that it is spliced out by the cellular machinery (e.g., the splicesome). Such a construct can be introduced into one or more plant cells to reduce the phenotypic expression of a nucleic acid (e.g., a nucleic acid sequence that is normally expressed in a plant cell).

In some embodiments, a construct (e.g., an expression construct) can include an inverted-duplication of a segment of a TLA or CAO gene, where the inverted-duplication of the TLA or CAO gene segment includes a nucleotide sequence substantially identical to at least a portion of the TLA or CAO gene and the complement of the portion of the TLA or CAO gene, respectively. It would be appreciated that a single promoter can be used to drive expression of the inverted-duplication of the TLA or CAO gene segment, and that the inverted-duplication typically contains at least one copy of the portion of the TLA or CAO gene in the sense orientation. Such a construct can be introduced into one or more plant cells to delay, inhibit or otherwise reduce the expression of a TLA or CAO gene in the plant cells.

Representative RNAi nucleic acid molecules directed toward TLA2, TLA3 and TLA4 are shown in SEQ ID NOs: 27, 28 and 29, respectively, and a representative RNAi nucleic acid molecule directed toward CAO2, CAO3 and CAO4 is shown in SEQ ID NO:30. The sense strand and antisense strand are identified with dashed underlining, and a spacer or loop sequence lies between. It would be appreciated by the skilled artisan that the region of complementarity, between the antisense strand of the RNAi and the mRNA or between the antisense strand of the RNAi and the sense strand of the RNAi, can be over the entire length of the RNAi nucleic acid molecule, or the region of complementarity can be less than the entire length of the RNAi nucleic acid molecule. For example, a region of complementarity can refer to, for example, at least 15 nucleotides in length up to, for example, 500 nucleotides in length (e.g., at least 15, 16, 17, 18, 19, 20, 25, 28, 30, 35, 49, 50, 60, 75, 80, 100, 150, 180, 200, 250, 300, 320, 385, 420, 435 nucleotides in length up to, e.g., 30, 35, 36, 40, 45, 49, 50, 60, 65, 75, 80, 85, 90, 100, 175, 200, 225, 250, 280, 300, 325, 350, 400, 450, or 475 nucleotides in length). In some embodiments, a region of complementarity can refer to, for example, at least 15 contiguous nucleotides in length up to, for example, 500 contiguous nucleotides in length (e.g., at least 15, 16, 17, 18, 19, 20, 25, 28, 30, 35, 49, 50, 60, 75, 80, 100, 150, 180, 200, 250, 300, 320, 385, 420, 435 nucleotides in length up to, e.g., 30, 35, 36, 40, 45, 49, 50, 60, 65, 75, 80, 85, 90, 100, 175, 200, 225, 250, 280, 300, 325, 350, 400, 450, or 475 contiguous nucleotides in length).

It would be appreciated by the skilled artisan that complementary can refer to, for example, 100% sequence identity between the two nucleic acids. In addition, however, it also would be appreciated by the skilled artisan that complementary can refer to, for example, slightly less than 100% sequence identity (e.g., at least 95%, 96%, 97%, 98%, or 99% sequence identity). In calculating percent sequence identity, two nucleic acids are aligned and the number of identical matches of nucleotides (or amino acid residues) between the two nucleic acids (or polypeptides) is determined. The number of identical matches is divided by the length of the aligned region (i.e., the number of aligned nucleotides (or amino acid residues)) and multiplied by 100 to arrive at a percent sequence identity value. It will be appreciated that the length of the aligned region can be a portion of one or both nucleic acids up to the full-length size of the shortest nucleic acid. It also will be appreciated that a single nucleic acid can align with more than one other nucleic acid and hence, can have different percent sequence identity values over each aligned region.

The alignment of two or more nucleic acids to determine percent sequence identity can be performed using the computer program ClustalW and default parameters, which allows alignments of nucleic acid or polypeptide sequences to be carried out across their entire length (global alignment). Chenna et al., 2003, Nucleic Acids Res., 31(13):3497-500. ClustalW calculates the best match between a query and one or more subject sequences (nucleic acid or polypeptide), and aligns them so that identities, similarities and differences can be determined. Gaps of one or more residues can be inserted into a query sequence, a subject sequence, or both, to maximize sequence alignments. For fast pairwise alignment of nucleic acid sequences, the default parameters can be used (i.e., word size: 2; window size: 4; scoring method: percentage; number of top diagonals: 4; and gap penalty: 5); for an alignment of multiple nucleic acid sequences, the following parameters can be used: gap opening penalty: 10.0; gap extension penalty: 5.0; and weight transitions: yes. For fast pairwise alignment of polypeptide sequences, the following parameters can be used: word size: 1; window size: 5; scoring method: percentage; number of top diagonals: 5; and gap penalty: 3. For multiple alignment of polypeptide sequences, the following parameters can be used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; and residue-specific gap penalties: on. ClustalW can be run, for example, at the Baylor College of Medicine Search Launcher website or at the European Bioinformatics Institute website on the World Wide Web.

The skilled artisan also would appreciate that complementary can be dependent upon, for example, the conditions under which two nucleic acids hybridize. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY; Sections 7.37-7.57, 9.47-9.57, 11.7-11.8, and 11.45-11.57). Sambrook et al. disclose suitable Southern blot conditions for oligonucleotide probes less than about 100 nucleotides (Sections 11.45-11.46). The Tm between a nucleic acid that is less than 100 nucleotides in length and a second nucleic acid can be calculated using the formula provided in Section 11.46. Sambrook et al. additionally disclose Southern blot conditions for oligonucleotide probes greater than about 100 nucleotides (see Sections 9.47-9.54). The Tm between a nucleic acid greater than 100 nucleotides in length and a second nucleic acid can be calculated using the formula provided in Sections 9.50-9.51 of Sambrook et al.

The conditions under which membranes containing nucleic acids are prehybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed. Simply by way of example, high stringency conditions typically include a wash of the membranes in 0.2× SSC at 65° C.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane. A nucleic acid molecule is deemed to hybridize to a nucleic acid, but not to another nucleic acid, if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantified directly on a membrane or from an autoradiograph using, for example, a Phosphorlmager or a Densitometer (Molecular Dynamics, Sunnyvale, CA).

A construct (also known as a vector) containing a RNAi nucleic acid molecule is provided. Constructs, including expression constructs, are described herein and are known to those of skill in the art. Expression elements (e.g., promoters) that can be used to drive expression of a RNAi nucleic acid molecule are known in the art and include, without limitation, constitutive promoters such as, without limitation, the cassava mosaic virus (CsMVM) promoter, the cauliflower mosaic virus (CaMV) 35S promoter, the actin promoter, or the glyceraldehyde-3-phosphate dehydrogenase promoter, or tissue-specific promoters such as, without limitation, root-specific promoters such as the putrescine N-methyl transferase (PMT) promoter or the quinolinate phosphosibosyltransferase (QPT) promoter. It would be understood by a skilled artisan that a sense strand and an antisense strand can be delivered to and expressed in a target cell on separate constructs, or the sense and antisense strands can be delivered to and expressed in a target cell on a single construct (e.g., in one transcript). As discussed herein, a RNAi nucleic acid molecule delivered and expressed on a single strand also can include a spacer nucleic acid (e.g., a loop nucleic acid) such that the RNAi forms a small hairpin (shRNA).

### Transgenic Plants and Methods of Making Transgenic Plants

Transgenic N. tabacum plants are provided that contain a transgene encoding at least one RNAi molecule, which, when transcribed, silences expression of any of the TLA or CAO sequences described herein. As used herein, silencing can refer to complete elimination or essentially complete elimination of the TLA or CAO mRNA, resulting in 100% or essentially 100% reduction (e.g., greater than 95% reduction; e.g., greater than 96%, 97%, 98% or 99% reduction) in the amount of encoded TLA or CAO polypeptide; silencing also can refer to partial elimination of the TLA or CAO mRNA (e.g., eliminating about 5%, 10%, 15%, 20%, 25%, 30%, 35%, 40%, 45%, 50% or more of the TLA or CAO mRNA), resulting in a reduction (e.g., about 5%, 10%, 15%, 20%, 25%, 30%, 35%, 40%, 45%, 50% or more, but not complete elimination) in the amount of the encoded TLA or CAO polypeptide.

A RNAi nucleic acid molecule can be transcribed using a plant expression vector. Methods of introducing a nucleic acid (e.g., a heterologous nucleic acid) into plant cells (e.g., N. tabacum cells) are known in the art and include, for example, particle bombardment, Agrobacterium-mediated transformation, microinjection, polyethylene glycol-mediated transformation (e.g., of protoplasts, see, for example, Yoo et al. (2007, Nature Protocols, 2(7):1565-72)), liposome-mediated DNA uptake, or electroporation.

Following transformation, the transgenic plant cells can be regenerated into transgenic tobacco plants. The regenerated transgenic plants can be screened for the presence of the transgene (e.g., a RNAi nucleic acid molecule) and/or one or more of the resulting phenotypes (e.g., reduced amount of TLA or CAO mRNA; reduced amount of TLA or CAO polypeptide; reduced activity of a TLA or CAO polypeptide; reduced concentration of thylakoid membranes in the photosystems; reduced amount of total chlorophyll; increased ratio of chlorophyll a to chlorophyll b; and/or increased biomass).

Methods of detecting alkaloids (e.g., nicotine) or TSNAs, and methods of determining the amount of one or more alkaloids or TSNAs are known in the art. For example, high performance liquid chromatography (HPLC)-mass spectroscopy (MS) (HPLC-MS) or high performance thin layer chromatography (HPTLC) can be used to detect the presence of one or more alkaloids and/or determine the amount of one or more alkaloids. In addition, any number of chromatography methods (e.g., gas chromatography/thermal energy analysis (GC/TEA), liquid chromatography/mass spectrometry (LC/MS), and ion chromatography (IC)) can be used to detect the presence of one or more TSNAs and/or determine the amount of one or more TSNAs.

As used herein, “reduced” or “reduction” refers to a decrease (e.g., a statistically significant decrease), in green leaf or cured leaf, of/in one or more of the following: a) the amount of TLA or CAO mRNA; b) the amount of TLA or CAO polypeptide; c) the activity of a TLA or CAO polypeptide; d) the concentration of thylakoid membranes in the photosystems measured spectrophotometrically from the amplitude of the light-minus-dark absorbance difference signal at 800 nm (P800) for PSI and 320 nm (QA) for PSII (see, for example, Melis & Brown, 1980, PNAS USA, 77(8):4712-6; and Melis, 1989, Philos. Trans. R. Soc. Lond. B, 323:397-409); and/or e) the amount of total chlorophyll. As used herein, “reduced” or “reduction” refers to a decrease in any of the above by at least about 5% up to about 95% (e.g., about 5% to about 10%, about 5% to about 20%, about 5% to about 50%, about 5% to about 75%, about 10% to about 25%, about 10% to about 50%, about 10% to about 90%, about 20% to about 40%, about 20% to about 60%, about 20% to about 80%, about 25% to about 75%, about 50% to about 75%, about 50% to about 85%, about 50% to about 95%, and about 75% to about 95%) relative to similarly-treated leaf (e.g., green or cured) from a tobacco plant lacking the transgene. As used herein, statistical significance refers to a p-value of less than 0.05, e.g., a p-value of less than 0.025 or a p-value of less than 0.01, using an appropriate measure of statistical significance, e.g., a one-tailed two sample t-test.

As used herein, “increased” refers to an increase (e.g., a statistically significant increase), in green leaf or cured leaf, of the ratio of chlorophyll a/chlorophyll b or in plant biomass. As used herein, “increased” refers to an increase in any of the above by at least about 5% up to about 95% (e.g., about 5% to about 10%, about 5% to about 20%, about 5% to about 50%, about 5% to about 75%, about 10% to about 25%, about 10% to about 50%, about 10% to about 90%, about 20% to about 40%, about 20% to about 60%, about 20% to about 80%, about 25% to about 75%, about 50% to about 75%, about 50% to about 85%, about 50% to about 95%, and about 75% to about 95%) relative to similarly-treated leaf (e.g., green or cured) from a tobacco plant lacking the transgene. As used herein, statistical significance refers to a p-value of less than 0.05, e.g., a p-value of less than 0.025 or a p-value of less than 0.01, using an appropriate measure of statistical significance, e.g., a one-tailed two sample t-test.

Leaf from progeny plants also can be screened for the presence of the transgene and/or the resulting phenotype, and plants exhibiting the desired phenotype can be selected. As described herein, leaf from such transgenic plants can exhibit reduced amount of TLA or CAO mRNA; reduced amount of TLA or CAO polypeptide; reduced activity of a TLA or CAO polypeptide; reduced concentration of thylakoid membranes in the photosystems; reduced amount of total chlorophyll; increased ratio of chlorophyll a to chlorophyll b; and/or increased biomass in the plant (e.g., compared to leaf from a plant lacking or not transcribing the RNAi). Leaf from regenerated transgenic plants can be screened (e.g., reduced amount of TLA or CAO mRNA; reduced amount of TLA or CAO polypeptide; reduced activity of a TLA or CAO polypeptide; reduced concentration of thylakoid membranes in the photosystems; reduced amount of total chlorophyll; increased ratio of chlorophyll a to chlorophyll b; and/or increased biomass), and the desired plants (e.g., having leaf that exhibit reduced amount of TLA or CAO mRNA; reduced amount of TLA or CAO polypeptide; reduced activity of a TLA or CAO polypeptide; reduced concentration of thylakoid membranes in the photosystems; reduced amount of total chlorophyll; increased ratio of chlorophyll a to chlorophyll b; and/or increased biomass), compared to the amount in a leaf from a corresponding non-transgenic plant, can be selected and, for example, used in a breeding program.

Transgenic plants exhibiting the desired phenotype can be used, for example, in a breeding program. Breeding is carried out using known procedures. Successful crosses yield F₁ plants that are fertile and that can be backcrossed with one of the parents if desired. In some embodiments, a plant population in the F₂ generation is screened for the presence of a transgene and/or the resulting phenotype using standard methods (e.g., amplification, hybridization and/or chemical analysis of the leaf). Selected plants are then crossed with one of the parents and the first backcross (BC₁) generation plants are self-pollinated to produce a BC₁F₂ population that is again screened. The process of backcros sing, self-pollination, and screening is repeated, for example, at least four times until the final screening produces a plant that is fertile and reasonably similar to the recurrent parent. This plant, if desired, is self-pollinated and the progeny are subsequently screened again to confirm that the plant contains the transgene and exhibits variant gene expression. Breeder's seed of the selected plant can be produced using standard methods including, for example, field testing and/or chemical analyses of leaf (e.g., cured leaf).

The result of a plant breeding program using the transgenic tobacco plants described herein are novel and useful varieties, lines, and hybrids. As used herein, the term “variety” refers to a population of plants that share constant characteristics which separate them from other plants of the same species. A variety is often, although not always, sold commercially. While possessing one or more distinctive traits, a variety is further characterized by a very small overall variation between individual with that variety. A “pure line” variety may be created by several generations of self-pollination and selection, or vegetative propagation from a single parent using tissue or cell culture techniques. A “line,” as distinguished from a variety, most often denotes a group of plants used non-commercially, for example, in plant research. A line typically displays little overall variation between individuals for one or more traits of interest, although there may be some variation between individuals for other traits.

A variety can be essentially derived from another line or variety. As defined by the International Convention for the Protection of New Varieties of Plants (Dec. 2, 1961, as revised at Geneva on Nov. 10, 1972, On Oct. 23, 1978, and on Mar. 19, 1991), a variety is “essentially derived” from an initial variety if: a) it is predominantly derived from the initial variety, or from a variety that is predominantly derived from the initial variety, while retaining the expression of the essential characteristics that result from the genotype or combination of genotypes of the initial variety; b) it is clearly distinguishable from the initial variety; and c) except for the differences which result from the act of derivation, it conforms to the initial variety in the expression of the essential characteristics that result from the genotype or combination of genotypes of the initial variety. Essentially derived varieties can be obtained, for example, by the selection of a natural or induced mutant, a somaclonal variant, a variant individual plant from the initial variety, backcrossing, or transformation.

Hybrid tobacco varieties can be produced by preventing self-pollination of female parent plants (i.e., seed parents) of a first variety, permitting pollen from male parent plants of a second variety to fertilize the female parent plants, and allowing F₁ hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by cytoplasmic male sterility (CMS), nuclear male sterility, genetic male sterility, molecular male sterility where a transgene inhibits microsporogenesis and/or pollen formation, or self-incompatibility. Female parent plants having CMS are particularly useful. In embodiments in which the female parent plants are CMS, the male parent plants typically contain a fertility restorer gene to ensure that the F₁ hybrids are fertile. In other embodiments in which the female parents are CMS, male parents can be used that do not contain a fertility restorer. F₁ hybrids produced from such parents are male sterile. Male sterile hybrid seed can be interplanted with male fertile seed to provide pollen for seed-set on the resulting male sterile plants.

Varieties and lines described herein can be used to form single-cross tobacco F₁ hybrids. In such embodiments, the plants of the parent varieties can be grown as substantially homogeneous adjoining populations to facilitate natural cross-pollination from the male parent plants to the female parent plants. The F₂ seed formed on the female parent plants is selectively harvested by conventional means. One also can grow the two parent plant varieties in bulk and harvest a blend of F₁ hybrid seed formed on the female parent and seed formed upon the male parent as the result of self-pollination. Alternatively, three-way crosses can be carried out wherein a single-cross F₁ hybrid is used as a female parent and is crossed with a different male parent. As another alternative, double-cross hybrids can be created wherein the F₁ progeny of two different single-crosses are themselves crossed. Self-incompatibility can be used to particular advantage to prevent self-pollination of female parents when forming a double-cross hybrid.

The tobacco plants used in the methods described herein can include, but are not limited to, a Burley type, a dark type, a flue-cured type, or an Oriental type. The tobacco plants used in the methods described herein typically are from N. tabacum, and can be from any number of N. tabacum varieties. A variety can be BU 64, CC 101, CC 200, CC 13, CC 27, CC 33, CC 35,CC 37, CC 65, CC 67, CC 301, CC 400, CC 500, CC 600, CC 700, CC 800, CC 900, CC 1063, Coker 176, Coker 319, Coker 371 Gold, Coker 48, CU 263, DF911, Galpao tobacco, GL 26H, GL 338, GL 350, GL 395, GL 600, GL 737, GL 939, GL 973, GF 157, GF₃₁₈, RJR 901, HB 04P, K 149, K 326, K 346, K 358, K394, K 399, K 730, NC 196, NC 37NF, NC 471, NC 55, NC 92, NC2326, NC 95, NC 925, PVH 1118, PVH 1452, PVH 2110, PVH 2254, PVH 2275, VA 116, VA 119, KDH 959, KT 200, KT204LC, KY 10, KY 14, KY 160, KY 17, KY 171, KY 907, KY907LC, KTY14×L8 LC, Little Crittenden, McNair 373, McNair 944, msKY 14×L8, Narrow Leaf Madole, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, Perique tobacco, PVH03, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN9OLC, TN 97, TN97LC, TN D94, TN D950, TR (Tom Rosson) Madole, VA 309, or VA359.

### Mutant Plants and Methods of Making

Methods of making a N. tabacum plant having a mutation are known in the art. Mutations can be random mutations or targeted mutations. For random mutagenesis, cells (e.g., N. tabacum cells) typically are mutagenized using, for example, a chemical mutagen or ionizing radiation. Representative chemical mutagens include, without limitation, nitrous acid, sodium azide, acridine orange, ethidium bromide, and ethyl methane sulfonate (EMS), while representative ionizing radiation includes, without limitation, x-rays, gamma rays, fast neutron irradiation, and UV irradiation. The dosage of the mutagenic chemical or radiation is determined experimentally for each type of plant tissue such that a mutation frequency is obtained that is below a threshold level characterized by lethality or reproductive sterility. The number of M₁ generation seed or the size of M₁ plant populations resulting from the mutagenic treatments are estimated based on the expected frequency of mutations. For targeted mutagenesis, representative technologies include TALEN (see, for example, Li et al., 2011, Nucleic Acids Res., 39(14):6315-25) or zinc-finger (see, for example, Wright et al., 2005, The Plant J., 44:693-705). Whether random or targeted, a mutation can be a point mutation, an insertion, a deletion, a substitution, or combinations thereof, which are discussed in more detail below.

The resultant variety of Nicotiana tabacum includes plants having a mutation in an endogenous TLA nucleic acid (e.g., SEQ ID NOs: 11, 13, 15, 17 or 19) encoding a TLA polypeptide sequence (e.g., SEQ ID NOs: 12, 14, 16, 18 or 20) or in an endogenous CAO gene (e.g., SEQ ID NOs: 21, 23 or 25) encoding a CAO polypeptide sequence (e.g., SEQ ID NOs: 22, 24 or 26). A mutation in a TLA or CAO sequence as described herein typically results in reduced expression or activity of TLA or CAO, which, in turn, results in one or more of the phenotypes described herein (e.g., reduced concentration of thylakoid membranes in the photosystems; reduced amount of total chlorophyll; increased ratio of chlorophyll a to chlorophyll b; and/or increased biomass), or combinations thereof depending on the particular combination of sequences that are mutated or otherwise knocked-down, in the leaf of a mutant plant relative to a plant lacking the mutation.

As discussed herein, one or more nucleotides can be mutated to alter the expression and/or function of the encoded polypeptide, relative to the expression and/or function of the corresponding wild type polypeptide. It will be appreciated, for example, that a mutation in one or more of the highly conserved regions would likely alter polypeptide function, while a mutation outside of those highly conserved regions would likely have little to no effect on polypeptide function. In addition, a mutation in a single nucleotide can create a stop codon, which would result in a truncated polypeptide and, depending on the extent of truncation, loss of function.

Suitable types of mutations in a TLA or CAO coding sequence include, without limitation, insertions of nucleotides, deletions of nucleotides, or transitions or transversions relative to the wild-type TLA or CAO coding sequence, respectively. Mutations in the coding sequence can result in insertions of one or more amino acids, deletions of one or more amino acids, conservative or non-conservative amino acid substitutions in the encoded polypeptide, or truncation of the protein (e.g., by introduction of a stop codon). In some cases, the coding sequence of a TLA comprises more than one mutation and/or more than one type of mutation.

Insertion or deletion of amino acids in a coding sequence, for example, can disrupt the conformation of the encoded polypeptide. Amino acid insertions or deletions also can disrupt sites important for recognition of binding ligand(s) or substrate(s) or for activity of the polypeptide. It is known in the art that the insertion or deletion of a larger number of contiguous amino acids is more likely to render the gene product non-functional, compared to a smaller number of inserted or deleted amino acids. In addition, one or more mutations (e.g., a point mutation) can change the localization of the TLA or CAO polypeptide, introduce a stop codon to produce a truncated polypeptide, or disrupt an active site or domain (e.g., a catalytic site or domain, a binding site or domain) within the polypeptide.

A “conservative amino acid substitution” is one in which one amino acid residue is replaced with a different amino acid residue having a similar side chain (see, for example, Dayhoff et al. (1978, in Atlas of Protein Sequence and Structure, 5(Suppl. 3):345-352), which provides frequency tables for amino acid substitutions), and a non-conservative substitution is one in which an amino acid residue is replaced with an amino acid residue that does not have a similar side chain. Non-conservative amino acid substitutions can replace an amino acid of one class with an amino acid of a different class. Non-conservative substitutions can make a substantial change in the charge or hydrophobicity of the gene product. Non-conservative amino acid substitutions can also make a substantial change in the bulk of the residue side chain, e.g., substituting an alanine residue for an isoleucine residue. Examples of non-conservative substitutions include a basic amino acid for a non-polar amino acid, or a polar amino acid for an acidic amino acid.

Following mutagenesis, Mo plants are regenerated from the mutagenized cells and those plants, or a subsequent generation of that population (e.g., M₁, M₂, M₃, etc.), can be screened for those carrying a mutation in a TLA of CAO sequence. Screening for plants carrying a mutation in a TLA of CAO nucleic acid or polypeptide can be performed directly using methods routine in the art (e.g., hybridization, amplification, nucleic acid sequencing, peptide sequencing, combinations thereof) or by evaluating the phenotype (e.g., reduced amount of TLA or CAO mRNA; reduced amount of TLA or CAO polypeptide; reduced activity of a TLA or CAO polypeptide; reduced concentration of thylakoid membranes in the photosystems; reduced amount of total chlorophyll; increased ratio of chlorophyll a to chlorophyll b; and/or increased biomass). It would be understood that the phenotype of a mutant plant (e.g., reduced amount of TLA or CAO mRNA; reduced amount of TLA or CAO polypeptide; reduced activity of a TLA or CAO polypeptide; reduced concentration of thylakoid membranes in the photosystems; reduced amount of total chlorophyll; increased ratio of chlorophyll a to chlorophyll b; and/or increased biomass) would be compared to a corresponding plant (e.g., having the same varietal background) that lacks the mutation.

An M₁ tobacco plant may be heterozygous for a mutant allele and exhibit a wild type phenotype. In such cases, at least a portion of the first generation of self-pollinated progeny of such a plant exhibits a wild type phenotype. Alternatively, an M₁ tobacco plant may have a mutant allele and exhibit a mutant phenotype (e.g., reduced amount of TLA or CAO mRNA; reduced amount of TLA or CAO polypeptide; reduced activity of a TLA or CAO polypeptide; reduced concentration of thylakoid membranes in the photosystems; reduced amount of total chlorophyll; increased ratio of chlorophyll a to chlorophyll b; and/or increased biomass). Such plants may be heterozygous and exhibit a mutant phenotype due to a phenomenon such as dominant negative suppression, despite the presence of the wild type allele, or such plants may be homozygous due to independently induced mutations in both alleles.

As used herein, “reduced” or “reduction” refers to a decrease (e.g., a statistically significant decrease), in green leaf or cured leaf, of/in one or more of the following: a) the amount of TLA or CAO mRNA; b) the amount of TLA or CAO polypeptide; c) the activity of a TLA or CAO polypeptide; d) the concentration of thylakoid membranes in the photosystems measured spectrophotometrically from the amplitude of the light-minus-dark absorbance difference signal at 800 nm (P800) for PSI and 320 nm (QA) for PSII (see, for example, Melis & Brown, 1980, PNAS USA, 77(8):4712-6; and Melis, 1989, Philos. Trans. R. Soc. Lond. B, 323:397-409); and/or e) the amount of total chlorophyll. As used herein, “reduced” or “reduction” refers to a decrease in any of the above by at least about 5% up to about 95% (e.g., about 5% to about 10%, about 5% to about 20%, about 5% to about 50%, about 5% to about 75%, about 10% to about 25%, about 10% to about 50%, about 10% to about 90%, about 20% to about 40%, about 20% to about 60%, about 20% to about 80%, about 25% to about 75%, about 50% to about 75%, about 50% to about 85%, about 50% to about 95%, and about 75% to about 95%) relative to similarly-treated leaf (e.g., green or cured) from a tobacco plant lacking the transgene. As used herein, statistical significance refers to a p-value of less than 0.05, e.g., a p-value of less than 0.025 or a p-value of less than 0.01, using an appropriate measure of statistical significance, e.g., a one-tailed two sample t-test.

As used herein, “increased” refers to an increase (e.g., a statistically significant increase), in green leaf or cured leaf, of the ratio of chlorophyll a/chlorophyll b or in plant biomass. As used herein, “increased” refers to an increase in any of the above by at least about 5% up to about 95% (e.g., about 5% to about 10%, about 5% to about 20%, about 5% to about 50%, about 5% to about 75%, about 10% to about 25%, about 10% to about 50%, about 10% to about 90%, about 20% to about 40%, about 20% to about 60%, about 20% to about 80%, about 25% to about 75%, about 50% to about 75%, about 50% to about 85%, about 50% to about 95%, and about 75% to about 95%) relative to similarly-treated leaf (e.g., green or cured) from a tobacco plant lacking the transgene. As used herein, statistical significance refers to a p-value of less than 0.05, e.g., a p-value of less than 0.025 or a p-value of less than 0.01, using an appropriate measure of statistical significance, e.g., a one-tailed two sample t-test.

A tobacco plant carrying a mutant allele can be used in a plant breeding program to create novel and useful lines, varieties and hybrids. Desired plants that possess the mutation can be backcrossed or self-pollinated to create a second population to be screened. Backcrossing or other breeding procedures can be repeated until the desired phenotype of the recurrent parent is recovered. DNA fingerprinting, SNP or similar technologies may be used in a marker-assisted selection (MAS) breeding program to transfer or breed mutant alleles into other tobaccos, as described herein.

In some embodiments, an M₁, M₂, M₃ or later generation tobacco plant containing at least one mutation is crossed with a second Nicotiana tabacum plant, and progeny of the cross are identified in which the mutation(s) is present. It will be appreciated that the second Nicotiana tabacum plant can be one of the species and varieties described herein. It will also be appreciated that the second Nicotiana tabacum plant can contain the same mutation as the plant to which it is crossed, a different mutation, or be wild type at the locus. Additionally or alternatively, a second tobacco line can exhibit a phenotypic trait such as, for example, disease resistance, high yield, high grade index, curability, curing quality, mechanical harvesting, holding ability, leaf quality, height, plant maturation (e.g., early maturing, early to medium maturing, medium maturing, medium to late maturing, or late maturing), stalk size (e.g., small, medium, or large), and/or leaf number per plant (e.g., a small (e.g., 5-10 leaves), medium (e.g., 11-15 leaves), or large (e.g., 16-21) number of leaves).

### Cured Tobacco and Tobacco Products

The methods described herein allow for increasing tobacco biomass while still maintaining high leaf quality. As described herein, such methods can include the production of transgenic (using, e.g., RNAi or overexpression) or mutant (e.g., random or targeted) plants.

Leaf quality can be determined, for example, using an Official Standard Grade published by the Agricultural Marketing Service of the US Department of Agriculture (7 U.S.C. §511); Legacy Tobacco Document Library (Bates Document #523267826/7833, Jul. 1, 1988, Memorandum on the Proposed Burley Tobacco Grade Index); and Miller et al., 1990, Tobacco Intern., 192:55-7. For dark-fired tobacco, leaves typically are obtained from stalk position C, and the average grade index determined based on Federal Grade and 2004 Price Support for Type 23 Western dark-fired tobacco.

Leaf from the tobacco described herein can be cured, aged, conditioned, and/or fermented. Methods of curing tobacco are well known and include, for example, air curing, fire curing, flue curing and sun curing. Aging also is known and is typically carried out in a wooden drum (e.g., a hogshead) or cardboard cartons in compressed conditions for several years (e.g., 2 to 5 years), at a moisture content of from about 10% to about 25% (see, for example, U.S. Pat. No. 4,516,590 and 5,372,149). Conditioning includes, for example, a heating, sweating or pasteurization step as described in US 2004/0118422 or US 2005/0178398, while fermenting typically is characterized by high initial moisture content, heat generation, and a 10 to 20% loss of dry weight. See, e.g., U.S. Pat. Nos. 4,528,993; 4,660,577; 4,848,373; and 5,372,149. The tobacco also can be further processed (e.g., cut, expanded, blended, milled or comminuted), if desired, and used in a tobacco product.

Tobacco products are known in the art and include any product made or derived from tobacco that is intended for human consumption, including any component, part, or accessory of a tobacco product. Representative tobacco products include, without limitation, cigarettes, smokeless tobacco products, tobacco-derived nicotine products, cigarillos, non-ventilated recess filter cigarettes, vented recess filter cigarettes, cigars, snuff, electronic cigarettes, e-vapor products, pipe tobacco, cigar tobacco, cigarette tobacco, chewing tobacco, leaf tobacco, shredded tobacco, and cut tobacco. Representative smokeless tobacco products include, for example, chewing tobacco, snus, pouches, films, tablets, sticks, rods, and the like. Representative cigarettes and other smoking articles include, for example, smoking articles that include filter elements or rod elements, where the rod element of a smokeable material can include cured tobacco within a tobacco blend. In addition to the reduced-nicotine or reduced-TSNA tobacco described herein, tobacco products also can include other ingredients such as, without limitation, binders, plasticizers, stabilizers, and/or flavorings. See, for example, US 2005/0244521, US 2006/0191548, US 2012/0024301, US 2012/0031414, and US 2012/0031416 for examples of tobacco products.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Sampling, RNA Preparation and Sequencing

Tobacco leaf sample from a burley variety, TN90, was collected. RNA from the sample was isolated using RNeasy Plant Mini Kit (Qiagen; MA) and its quality tested using Agilent Plant RNA Nano Kit and a 2100 Bioanalyzer (Agilent Technologies, CA, USA). A cDNA library was constructed and indexed using a TrueSeq RNA Library Prep Kit v.2 (Illumina). cDNAs were run on an Illumina HiSeq 2000 under conditions for 100 bp single reads and a minimum of 30 million reads per sample. Leaf gene expression in TN90 tobacco was determined by RNA deep sequencing performed by ArrayXpress (Raleigh, NC).

### Example 2

### Tobacco TLA2, TLA3 and TLA4 Homologous Gene Identification, Full Length Gene Cloning

TLA2, TLA3 and TLA4 gene sequences from Chlamydomonas and Arabidopsis were used to Blast the TN90 burley genomic sequence data base and leaf RNA sequence data. Five candidate genes were identified: TLA2, TLA2 Homo, TLA3, TLA3 Homo, and TLA4, and primers were designed to clone the five candidate genes. Leaf tissue was collected and a cDNA library was created using the In-Fusion® SMARTer® Directional cDNA Library Construction Kit from Clontech. Full length candidate genes were amplified using the gene specific primers designed from predicted full length cDNA sequences. The full length coding sequences were identified, cloned and confirmed by sequencing.

TABLE 1
Primers for TLA gene cloning

| | | SEQ |
|---|---|---|
| | | ID |
| Designation | SEQUENCE (5′ to 3′) | NO |
| TLA-2-F | ATGGCTTCTCTATTATCTTCTCGTC | 1 |
| TLA-2-R | TCATGGGAAGATAGCATTAACAAA | 2 |
| TLA-2-Homo-F | ATGGCTTCTCTATTATCTTCTCGTCTCC | 3 |
| TLA-2-Homo-R | TTAAATAGAATCTGCCACATAAATG | 4 |
| TLA-3-F | ATGGATGCTCTCTTCGTCAATTCCTC | 5 |
| TLA-3-R | GCCCTTGTTCAACTTACTACATT | 6 |
| TLA-3-Homo-F | ATGGATGCTCTGTTCGTCAATTCCT | 7 |
| TLA-3-Homo-R | TCAACTTACTACATTCTGTTGACCC | 8 |
| TLA-4-F | ATGGAAGCCACTGCTTCTTTCTCCTCA | 9 |
| TLA-4-R | TTAGTTCTTGGCCCCAAAACCACGAG | 10 |

The sequence of the candidate genes are provided as indicated in Table 2.

TABLE 2
Candidate genes

| Gene name | Nucleic Acid | Polypeptide |
|---|---|---|
| TLA2 | SEQ ID NO: 11 | SEQ ID NO: 12 |
| TLA2-Homo | SEQ ID NO: 13 | SEQ ID NO: 14 |
| TLA3 | SEQ ID NO: 15 | SEQ ID NO: 16 |
| TLA3-homo | SEQ ID NO: 17 | SEQ ID NO: 18 |
| TLA4 | SEQ ID NO: 19 | SEQ ID NO: 20 |
| CAO-2 | SEQ ID NO: 21 | SEQ ID NO: 22 |
| CAO-3 | SEQ ID NO: 23 | SEQ ID NO: 24 |
| CAO-4 | SEQ ID NO: 25 | SEQ ID NO: 26 |

### Example 3

### RNAi Plasmid Construction and Agrobacterium Transformation

In order to investigate the function of the candidate genes, RNAi constructs were produced against TLA2, TLA3 and TLA4, and transgenic plant lines were generated. An Agrobacterium expression vector (SEQ ID NO:31) was used, which has a CsVMV promoter and a NOS terminator, as well as a cassette having a kanamycin selection marker (NPT II) under direction of the actin2 promoter and a NOS terminator. The nucleic acid constructs carrying each RNAi construct were introduced into tobacco leaf disc using an Agrobacterium transformation approach. See, for example, Mayo et al., 2006, Nat Protoc., 1(3):1105-11; and Horsch et al., 1985, Science, 227:1229-31.

Briefly, ascetical tobacco plants (Narrow Leaf Madole (NLM)) were grown in magenta boxes, and leaf discs were cut onto 15×150 mm plates. Agrobacterium tumefaciens containing each nucleic acid construct were collected by centrifugation of 20 ml cell suspension in 50 ml centrifuge tube at 3500 rpm for 10 minutes. Supernatant was removed and the Agrobacterium cell pellet was re-suspended in 40 ml liquid re-suspension medium. About 25 ml of the solution was transferred to each 15×100 mm petri plates. In those 15×150 mm plates, tobacco leaves were cut into 0.6 cm discs (avoiding the midrib).

Leaves were placed upside down, a thin layer of MS/B5 liquid re-suspension medium was added, and leaf discs were produced using a #15 razor blade. The leaf discs were poked uniformly with a fine point needle. Eight discs were placed in each regeneration plate (15×100 mm). Agrobacterium tumefaciens suspension was added and incubated with the leaf discs for 10 minutes. Leaf discs were transferred to co-cultivation plates (1/2MS medium) and discs were placed upside down in contact with filter paper overlaid on the co-cultivation TOM medium (MS medium with 20 g sucrose/L; 1 mg/L IAA and 2.5 mg/L BAP). The plate was sealed with parafilm and labeled appropriately.

Plates were incubated in dim light (60-80 mE/ms) under 18/6 photoperiods at 24° C. for three days. Leaf discs were transferred to regeneration/selection medium plates with TOM K media (TOM medium with 300 mg/L kanamycin) and sub-cultured bi-weekly in the same fresh medium in dim light at 24° C. until shoots become excisable. Shoots from leaves were removed with forceps and inserted in MS basal medium with 100 mg/L kanamycin for rooting at 24° C. under 18/6 photoperiods in dim light (60-80 mE/ms). When plantlets having both shoots and roots grew large enough (e.g., reached over half of the height of the GA7 box), they were transferred to soil for acclimatization. During the transfer, any gel remaining on the root tissue was washed off with tap water. Established seedlings were transferred to the greenhouse to set seed and for further analysis.

An RNAi sequence against TLA2 is provided in SEQ ID NO:27. The sense, spacer and antisense portions of the RNAi molecule are provided in SEQ ID NOs: 45-47, respectively. An RNAi sequence against TLA3 is provided in SEQ ID NO:28. The sense, spacer and antisense portions of the RNAi molecule are provided in SEQ ID NOs: 48-50, respectively. An RNAi sequence against TLA4 is provided in SEQ ID NO:29. The sense, spacer and antisense portions of the RNAi molecule are provided in SEQ ID NOs: 51-53, respectively. An RNAi sequence against CAO-2, CAO-3 and CAO-4 is provided in SEQ ID NO:30. The sense, spacer and antisense portions of the RNAi molecule are provided in SEQ ID NOs: 54-56, respectively.

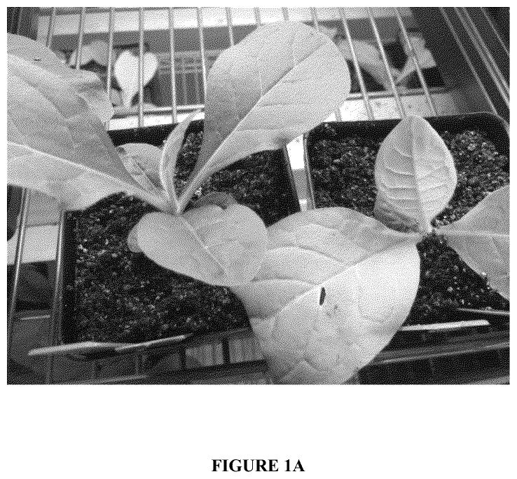

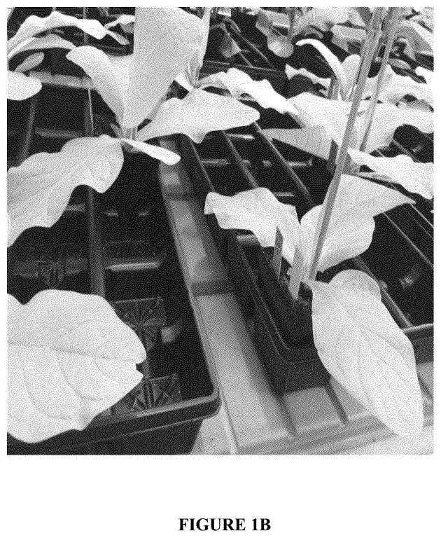

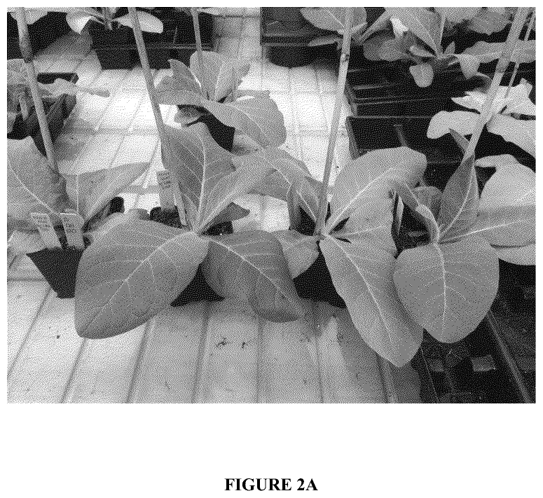

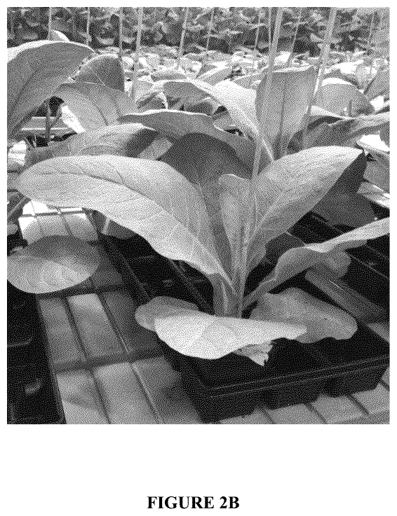

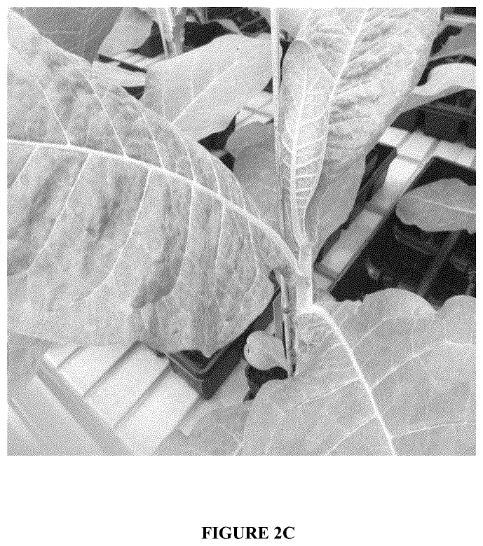

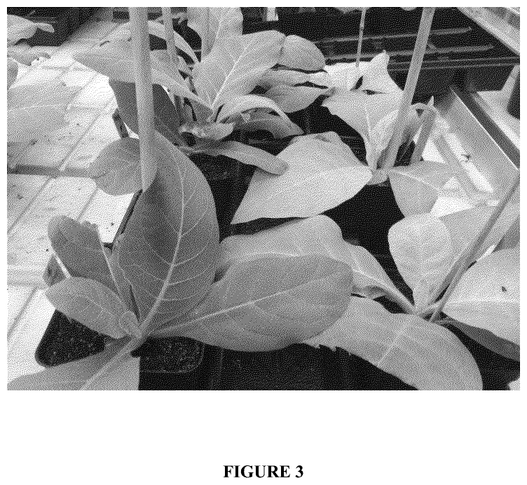

FIGS. 1, 2 and 3 are photographs of the transgenic plants described herein. FIG. 1A shows a T0 transgenic tobacco plant containing a TLA2 RNAi construct (right) next to a 15 wild type tobacco plant (left), while FIG. 1B shows a T1 transgenic tobacco plant containing a TLA2 RNAi construct. FIG. 2A shows T0 transgenic tobacco plants containing different integration events for a TLA3 RNAi construct (the three plants on the left) next to a wild type tobacco plant (the plant on the right). FIGS. 2B and 2C show two T1 transgenic tobacco plants containing a TLA3 RNAi construct. FIG. 3 shows transgenic tobacco plants containing a TLA4 RNAi construct (right) next to wild type tobacco plants (left).

FIGS. 1, 2 and 3 shows that transgenic tobacco plants expressing a RNAi construct against a TLA gene exhibit lower chlorophyll levels depends on the target gene and integration event. As described herein, chlorophyll levels can be qualitatively determined by observing the color of the leaf and/or quantitatively determined by measuring the amount of total chlorophyll and/or the ratio of chlorophyll a to chlorophyll b.

### Example 4

### Real Time PCR Confirmation and Western Blot Evaluation on TLA RNAi Transgenic Lines

RealTime PCR analysis: To confirm the expression pattern of selected candidate genes, relative changes in transcripts from 16 different samples were measured. In brief, total RNA was isolated using TRI Reagent (Sigma-Aldritch). To remove DNA impurities, purified RNA was treated with RNase free DNase (Turbo DNA-free; Ambion). To synthesize the first cDNA strand, approximately 10 μg of total RNA was transcribed utilizing the High Capacity cDNA Kit (Applied Biosystems). To measure the level of selected gene transcripts in the samples, RT PCR was performed using SYBR Green PCR Master Mix (Applied Biosystems). Gene specific primers are shown below.

Antigenic domains were identified from the sequences of the TLA2, TLA3 and TLA4 proteins. The oligopeptides shown in the Table below were synthesized and injected into rabbit to generate polyclonal antibodies. Western blots then were used to confirm the protein expression level for the target knock down genes.

Oligopeptides (solid underlining or dashed underlining represents different charges for the amino acids), and the ratio is the ratio of polar amino acids to total amino acids:

TLA2

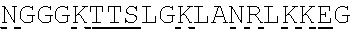

[SEQ ID NO: 32; corresponds to positions 178-197
of SEQ ID NO: 12] (11:20)

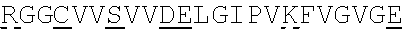

[SEQ ID NO: 33; corresponds to positions 330-352
of SEQ ID NO: 12] (7:23)
TLA3

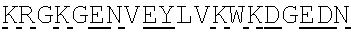

[SEQ ID NO: 34; corresponds to positions 275-294
of SEQ ID NO: 16] (13:20)

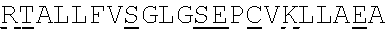

[SEQ ID NO: 35; corresponds to positions 158-179
of SEQ ID NO: 16] (8:22)
TLA4

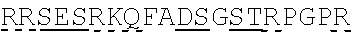

[SEQ ID NO: 36; corresponds to positions 538-557
of SEQ ID NO: 20] (14:20)

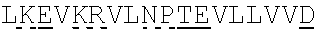

[SEQ ID NO: 37; corresponds to positions 279-296
of SEQ ID NO: 20] (8:18)

TABLE 3
Real time PCR primers list

| | | SEQ |
|---|---|---|
| | | ID |
| Designation | Sequence (5′ to 3′) | NO: |
| TLA2-F1 | ATGGCTTCTCTATTATCTTCTCGTCTC | 38 |
| TLA2-R1 | GTTCAAATGCTCAGCTGGTGGAACG | 39 |
| TLA3-F1 | CGTCAATTCCTCTCTCTCCCGCCTC | 40 |
| TLA3-R1 | CTTCAGAACCAGCAGCAACAAGCAG | 41 |
| TLA4-F1 | ATGGAAGCCACTGCTTCTTTCTCCTC | 42 |
| TLA4-R1 | CTCATTCACTAGGGATACATGGAGGGTG | 43 |

Total tobacco leaf protein extracts from TLA3-RNAi transgenic plants (e.g., TLA3-1, 691 WT, and TLA3-18) were loaded (on the basis of equal Ch1) and run on an SDS-PAGE gel. Western blotting was performed on the SDS-PAGE gel and the membrane was probed with specific polyclonal antibodies raised against two TLA3 oligopeptides (primary anti-TLA3 antibody diluted at 1:500). Substantially lower amounts of the TLA3 protein was observed in the extracts from the TLA3-1 and TLA3-18 plants compared to that of the wild type plants.

Interestingly, the TLA3 protein from N. tabacum has a predicted molecular weight of 35 kD, but, under electrophoretic conditions, the protein migrates to a position of about 25 kD. This faster electrophoretic mobility is attributed to the fact that TLA3 from N. tabacum has about 45 negatively charged amino acids and, thus, migrates faster under the influence of the electrophoresis field.

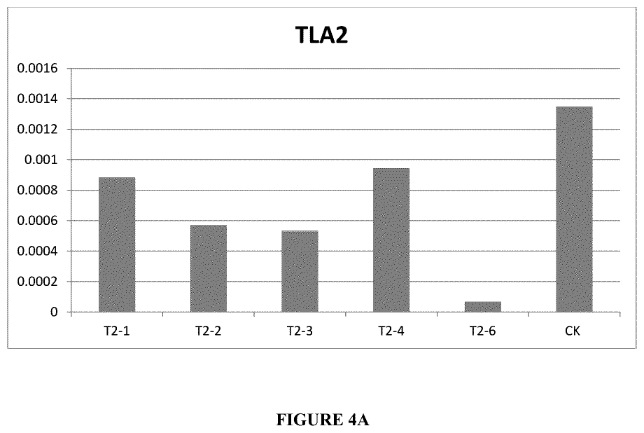

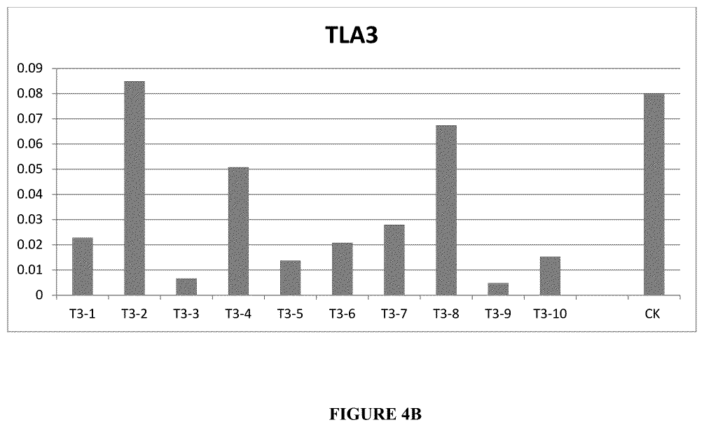

FIG. 4A is a graph showing the real time PCR results from T0 generation plants transgenic for TLA2 RNAi. The T2-1, T2-2, T2-3, T2-4 and T2-6 designations represent individual TLA2 RNAi transgenic plants while CK refers to a transgenic control tobacco plant (transformed with an empty vector). The Y axis (2\hat{ }-Δct) shows the relative expression level, based on mRNA levels, of the TLA2 gene. There were different levels of knock-down of the TLA2 gene depending upon the particular integration event. Among them, the T2-6 line exhibited plants with stunted growth; most of the plants died before they matured.

Overall, regenerating transgenic tobacco plants deficient in TAL2 was difficult, and the growth was inhibited by the level of TLA2 knock-down.

FIG. 4B is a graph showing the real time PCR results in T0 generation plants transgenic for TLA3 RNAi. T3-1 to T3-10 designations represent individual TLA3 RNAi transgenic plants, whereas CK refers to transgenic control tobacco (e.g., transformed with an empty vector). As with TLA2 transformants, there were different levels of knock down of TLA3 gene in TLA3 RNAi transformants.

### Example 5

### Phenotype Evaluation on TLA RNAi Transgenic Lines

According to the TLA concept proposed by the University of California - Berkeley group (Polle et al., 2003, Planta, 217:49-59; Kirst et al., 2012, Plant Physiol., 158:930-45; and Kirst et al., 2012, Plant Physiol., 160:2251-60), the mutants described herein should result in smaller light-harvesting chlorophyll antenna size and a substantially improved photosynthetic efficiency, as well as a higher [chlorophyll a/chlorophyll b] ratio. The chlorophyll from tobacco leaf samples was extracted in 80% acetone, and cell debris was removed by centrifugation at 20,000×g for 5 min. The absorbance of the supernatant was measured with a Shimadzu UV-1800 spectrophotometer, and the Chl concentration of the samples was determined according to Arnon (1949, Plant Physiol., 24:1-15), with equations corrected as described by Melis et al. (1989, Philos. Trans. R. Soc. Lond. B, 323:397-409). Total carotenoid content was determined according to the method of Lichtenthaler (1987, Methods Enzymol., 148:350-82).

The antenna size of tobacco leaf samples was calculated by measuring photosynthetic activity. The oxygen evolution activity of the tobacco leaf (punched from fresh leaf tissue sample) was measured at 25° C. with a Clark-type oxygen electrode illuminated with light from a halogen lamp projector. A Corning 3-69 filter (510-nm cutoff filter) defined the yellow actinic excitation via which photosynthesis measurements were made. Samples of 5-mL cell suspension containing 1.3 mM Chl were loaded into the oxygen electrode chamber. Sodium bicarbonate (100 mL of 0.5 M solution, pH 7.4) was added to the cell suspension prior to the oxygen evolution measurements to ensure that oxygen evolution was not limited by the carbon supply available to the cells. After registration of the rate of dark respiration by the cells, samples were illuminated with gradually increasing light intensities. The rate of oxygen exchange (uptake or evolution) under each of these irradiance conditions was recorded continuously for a period of about 5 min.

The following Table shows total chlorophyll measurement and Chlorophyll a/Chlorophyll b ratios of T0 lines transgenic for TLA2, TLA3 or TLA4 RNAi.

TABLE 4
Chlorophyll measurements

| | Chl a | Chl b | total Chl | Chl a/b |
|---|---|---|---|---|
| Sample | (μg/mL) | (μg/mL) | (μg/mL) | Ratio |
| BW1* | 17.96 | 4.29 | 22.240 | 4.190 |
| BM2* | 5.56 | 0.61 | 6.165 | 9.190 |
| Wild type | 43.07 | 11.40 | 54.465 | 3.780 |
| TLA2-1 | 29.90 | 5.97 | 35.860 | 5.012 |
| TLA2-2 | 26.40 | 6.56 | 32.950 | 4.027 |
| TLA2-3 | 5.93 | 1.29 | 7.215 | 4.615 |
| TLA2-4 | 20.34 | 4.68 | 25.015 | 4.351 |
| TLA2-5 | 50.73 | 13.85 | 64.570 | 3.664 |
| TLA2-6 | 18.78 | 4.76 | 23.535 | 3.944 |
| TLA2-7 | 24.09 | 5.55 | 29.640 | 4.341 |
| TLA3-1 | 29.53 | 6.66 | 36.185 | 4.433 |
| TLA3-2 | 45.05 | 11.00 | 56.040 | 4.097 |
| TLA3-3 | 43.16 | 6.10 | 49.255 | 7.081 |
| TLA3-4 | 36.73 | 9.04 | 45.770 | 4.063 |
| TLA3-5 | 38.05 | 7.22 | 45.265 | 5.274 |
| TLA3-6 | 27.72 | 5.01 | 32.720 | 5.537 |
| TLA3-7 | 30.27 | 5.27 | 35.540 | 5.744 |
| TLA3-8 | 53.82 | 13.29 | 67.105 | 4.049 |
| TLA3-9 | 29.61 | 4.95 | 34.555 | 5.988 |
| TLA3-10 | 24.09 | 4.45 | 28.535 | 5.420 |
| TLA3-11 | 36.04 | 5.31 | 41.340 | 6.793 |
| TLA3-12 | 28.50 | 4.27 | 32.770 | 6.674 |
| TLA3-13 | 33.61 | 5.63 | 39.235 | 5.969 |
| TLA3-14 | 22.65 | 3.75 | 26.400 | 6.040 |
| TLA3-15 | 21.62 | 3.29 | 24.910 | 6.571 |
| TLA3-16 | 27.80 | 5.77 | 33.565 | 4.817 |
| TLA3-17 | 33.03 | 6.90 | 39.920 | 4.790 |
| TLA3-18 | 27.34 | 7.35 | 34.690 | 3.720 |
| TLA3-19 | 30.06 | 6.67 | 36.725 | 4.510 |
| TLA3-20 | 23.56 | 4.44 | 27.995 | 5.305 |
| TLA3-21 | 21.25 | 2.89 | 24.135 | 7.366 |
| TLA3-22 | 22.28 | 4.17 | 26.450 | 5.343 |
| TLA4-1 | 8.90 | 2.07 | 10.960 | 4.308 |
| TLA4-2 | 46.74 | 12.06 | 58.790 | 3.877 |
| TLA4-3 | 29.90 | 5.97 | 35.860 | 5.012 |
| TLA4-4 | 21.04 | 4.83 | 25.870 | 4.356 |
| TLA4-5 | 39.16 | 8.72 | 47.875 | 4.493 |
| TLA4-6 | 38.63 | 7.88 | 46.505 | 4.902 |

*BW1: UC Berkley wild type line; BM2: UC Berkley TLA mutant line

Tobacco lines exhibit a particular ratio of chlorophyll a/b, which can be used as an index for total antenna size. Therefore, changes in the chlorophyll a/b ration can be used to measure changes in antenna size. As shown in the Table above, the UC Berkeley mutant line increased the chlorophyll a/b ratio to about 9, from a wild type ratio of about 4. In the NLM tobacco lines described herein, the wild type ratio of chlorophyll a/b is about 4, but in T0 generations of NLM plants transgenic for a TLA3 RNAi, most of the mutant lines reached a chlorophyll a/b ratio of about 5 to about 8. There was no obvious increase in chlorophyll a/b ratio in the T0 generation of plants transgenic for TLA2 RNAi and TLA4 RNAi. These results indicate that TLA3 likely is the initial candidate gene to knock down and decrease antenna size in photosynthetic light harvesting centers in tobacco.

T1 generation of TLA3 RNAi line 1 (501-1 to 501-24) and 3 (data not shown) were harvested and chlorophyll a/b ratios were determined. The following Table shows chlorophyll data for the T1 generation of the TLA3 RNAi plants. The chlorophyll a/b ratios in the T1 generation of the TLA3 RNAi plants were higher compared to wild type ratios, and the change in the ratio was stable in the T1 generation.

TABLE 5
Chlorophyll measurements

| | Chl a | Chl b | total Chl | Chl a/b |
|---|---|---|---|---|
| Sample | (μg/mL) | (μg/mL) | (μg/mL) | Ratio |
| 503_1 | 19.48 | 2.58 | 22.06 | 7.57 |
| 503_2 | 31.14 | 6.85 | 37.99 | 4.55 |
| 503_3 | 32.94 | 7.37 | 40.32 | 4.47 |
| 503_4 | 31.71 | 6.59 | 38.30 | 4.81 |
| 503_5 | 44.48 | 8.26 | 52.74 | 5.38 |
| 503_6 | 44.88 | 9.67 | 54.56 | 4.64 |
| 503_7 | 49.16 | 10.74 | 59.89 | 4.58 |
| 503_8 | 38.81 | 8.09 | 46.90 | 4.80 |
| 503_9 | 40.97 | 9.89 | 50.86 | 4.14 |
| 503_10 | 33.30 | 7.27 | 40.57 | 4.58 |
| 503_11 | 29.03 | 5.86 | 34.89 | 4.95 |
| 503_12 | 33.10 | 7.77 | 40.87 | 4.26 |
| 503_13 | 30.27 | 5.62 | 35.88 | 5.39 |
| 503_14 | 34.49 | 6.89 | 41.38 | 5.01 |
| 503_15 | 36.55 | 7.74 | 44.28 | 4.72 |
| 503_16 | 34.80 | 7.00 | 41.79 | 4.97 |
| 503_17 | 42.21 | 8.61 | 50.81 | 4.90 |
| 503_18 | 20.70 | 3.24 | 23.93 | 6.39 |
| 503_19 | 38.04 | 8.50 | 46.54 | 4.48 |
| 503_20 | 38.30 | 7.79 | 46.08 | 4.92 |
| 503_21 | 29.45 | 4.86 | 34.31 | 6.06 |
| 503_22 | 34.28 | 8.08 | 42.36 | 4.24 |
| 503_23 | 29.14 | 5.79 | 34.92 | 5.04 |
| 503_24 | 28.36 | 8.96 | 37.31 | 3.17 |
| Wild type | 46.61 | 13.67 | 60.27 | 3.41 |

Antenna size of the photosynthetic light harvesting centers (both PSI and PSII) were measured in the T1 generation for TLA3 RNAi plants, TLA2 RNAi plants, and TLA4 RNAi plants. The following Table shows the antenna size measurement of PSI and PSII in the T1 generation for four TLA2 RNAi line 2 plants (e.g., 2-1-1, 2-1-2, 2-1-3, and 2-1-4). The data showed that total antenna size in PSI for mutant lines was similar to wild type, but that PSII antenna size decreased in TLA2 knock out lines.

TABLE 6
Chlorophyll and antenna measurements

| | | | | | | | PSII | PSI |
|---|---|---|---|---|---|---|---|---|
| | | | | | | | antenna | antenna |
| | Chl a | Chl b | Total | Total | Chl a/b | Chl/Car | size | size |
| Sample | content | content | Chl | Car | ratio | ratio | (molecules | (molecule |
| number | (μg/ml) | (μg/ml) | (μg/ml) | (μg/ml) | (mol:mol) | (mol:mol) | in PSII) | in PSI) |
| WT 1 | 18.84 | 5.93 | 24.77 | 5.48 | 3.18 | 4.52 | 215 | 180 |
| | | | | | (2.70*) | (5.54*) | | |
| WT 2 | 25.37 | 7.94 | 33.30 | 6.64 | 3.20 | 5.02 | 185 | 197 |
| | | | | | (2.73*) | (5.98*) | | |
| 2-1-1 | 18.13 | 5.29 | 23.42 | 4.93 | 3.43 | 4.75 | 164 | 178 |
| | | | | | (3.02*) | (6.16*) | | |
| 2-1-2 | 11.59 | 3.81 | 15.40 | 2.57 | 3.04 | 6.00 | 125 | 168 |
| | | | | | (2.88*) | (6.56*) | | |
| 2-1-3 | 17.90 | 4.73 | 22.63 | 4.48 | 3.79 | 5.05 | 125 | 184 |
| | | | | | (3.02*) | (6.66*) | | |
| 2-1-4 | 12.23 | 4.26 | 16.48 | 2.76 | 2.87 | 5.98 | 125 | 166 |
| | | | | | (2.92*) | (6.24*) | | |

*ratios measured in isolated thylakoids

The antenna size for PSI and PSII in the Ti generation of TLA3 RNAi line 3 plants (e.g., 3-3-1, 3-3-2, 3-3-3, 3-3-4, 3-3-5, 3-3-6, and 3-3-7) is shown in the following Table. Notably, the total number of photoreceptor antenna was knocked down in the transgenic plants from 215 to 160 for PSII and from 195 to 160 for PSI. The decrease in antenna size was correlated with the increase in chlorophyll a/b ratio in the Ti generation.

TABLE 7
Chlorophyll and antenna measurements

| | | | | | | | PSII | PSI |
|---|---|---|---|---|---|---|---|---|
| | | | | | | | antenna | antenna |
| | | | | | | | size | size |
| | Chl a | Chl b | Tot Chl* | Total Car | Chl a/b | Chl/Car | (molecules | (molecules |
| Sample | [μg/ml] | [μg/ml] | [μg/ml] | [μg/ml] | (mol:mol) | (mol:mol) | in PSII) | in PSII) |
| WT 1 | 32.1 | 11 | 43.1 | 7.91 | 2.91 | 5.46 | 215 | 195 |
| WT 2 | 26.6 | 8.83 | 35.5 | 6.29 | 3.02 | 5.64 | | |
| 3-3-1 | 16.8 | 3.68 | 20.5 | 4.4 | 4.56 | 4.65 | 160 | 164 |
| 3-3-2 | 15.1 | 3.43 | 18.5 | 4.12 | 4.4 | 4.49 | | |
| 3-3-3 | 13.9 | 3.26 | 17.1 | 3.4 | 4.26 | 5.04 | | |
| 3-3-4 | 14.6 | 3.36 | 18 | 3.57 | 4.34 | 5.03 | | |
| 3-3-5 | 15.8 | 3.54 | 19.4 | 4.66 | 4.47 | 4.16 | 148 | 162 |
| 3-3-6 | 32.1 | 11 | 43.1 | 7.91 | 2.91 | 5.46 | 215 | 195 |
| 3-3-7 | 26.6 | 8.83 | 35.5 | 6.29 | 3.02 | 5.64 | | |

The following Table shows antenna size in PSI and PSII from T1 generation plants transgenic for TLA4 RNAi line 2 and line 6. This data demonstrated that total antenna size of the mutant lines was similar to that of the wild type in both PSI and PSII.

TABLE 8
Chlorophyll and antenna measurements

| | | | | | | | PSII | PSI |
|---|---|---|---|---|---|---|---|---|
| | | | Tot | Total | | | antenna | antenna |
| Sample | Chl a | Chl b | Chl* | Car | Chl a/b | Chl/Car | size | size |
| WT 1 | 24.1 | 8.0 | 32.0 | 5.40 | 3.00 | 5.86 | 210 | |
| WT 2 | 27.3 | 9.2 | 36.5 | 6.29 | 2.93 | 6.03 | 200 | 195 |
| TLA4-2-1 | 30.1 | 10.8 | 41.0 | 6.30 | 2.76 | 6.48 | | |
| TLA4-2-2 | 32.9 | 12.0 | 45.0 | 6.80 | 2.72 | 6.55 | 232 | |
| TLA4-2-4 | 29.3 | 11.1 | 40.5 | 6.10 | 2.63 | 6.59 | 190 | |
| TLA4-6-1 | 29.3 | 11.1 | 40.5 | 6.10 | 2.63 | 6.59 | 156 | 165 |
| TLA4-6-2 | 27.9 | 9.7 | 37.7 | 6.30 | 2.85 | 5.90 | 230 | |
| TLA4-6-3 | 25.7 | 10.2 | 36.0 | 5.40 | 2.52 | 6.64 | 185 | 145 |

### Example 6

### CAO RNAi Plant Generation

Tobacco plants transgenic for the CAO RNAi nucleic acid shown in SEQ ID NO:30 were produced as described herein, and chlorophyll a and chlorophyll b was measured as described herein. The data is shown in the following Table.

TABLE 9
Chlorophyll measurements

| | Chl A | Chl B | Total | Chl a/b |
|---|---|---|---|---|
| Sample | (μg/mL) | (μg/mL) | (μg/mL) | Ratio |
| Wild type | 17.56 | 4.42 | 21.97 | 3.97 |
| CAOi-1 | 15.75 | 2.49 | 18.23 | 6.33 |
| CAOi-2 | 12.73 | 1.78 | 14.50 | 7.16 |

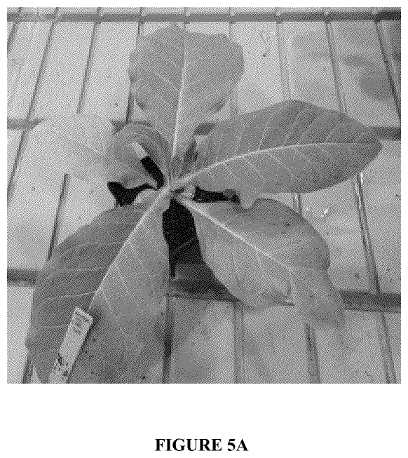

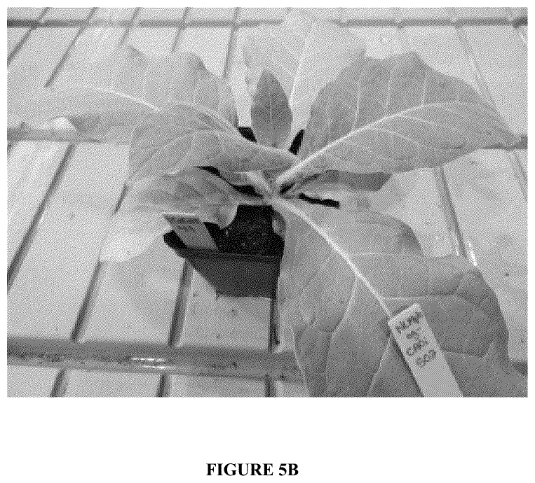

This data demonstrated that antenna size of the mutant lines was decreased relative to that of the wild type plants. FIG. 5A shows a T0 generation tobacco plant transgenic for CAOi-1, and FIG. 5B shows a T0 generation tobacco plant transgenic for CAOi-2.

### Example 7

### Phenotypes of TLA3 RNAi Plants

Decreasing, or truncating, the chlorophyll antenna size of the photosystems should improve photosynthetic solar energy conversion efficiency and productivity in mass cultures of algae or plants by up to 3-fold. A Truncated Light-harvesting chlorophyll Antenna size (TLA) in photosynthetic organisms should help alleviate excess absorption of sunlight and the ensuing wasteful non-photochemical dissipation of excitation energy and, thus, would increase solar-to-biomass energy conversion efficiency and photosynthetic productivity in high density cultures.

Tobacco was grown under conditions that result in high-density canopies to evaluate the TLA plants described herein. The T0 stage of multiple NL Madole TLA3-RNAi transformants were screened and selected to identify lines for generating T1 seeds. The latter were germinated and Ti leaves were subjected to phenotypic and functional analysis. Plants were grown in high density canopies, with the canopy layout of 25 plants in a 5×5 configuration, and the distance between individual plants set at 9 inches. Biochemical analysis and biomass accumulation was performed.

This work showed a 25% improvement in stem and leaf biomass accumulation for the TLA tobacco canopies over that of their wild-type counterparts grown under the same ambient conditions. Distinct differences were observed in the appearance of the canopy between plants containing a TLA RNAi and wild type tobacco plants. For example, the TLA3-RNAi canopy was a light-green color, while the wild type canopy was a much darker green. The results described herein can lead to significant improvements in agronomy, agricultural productivity, and the optimization of photosynthesis in commodity crops (e.g., tobacco) or parts thereof (e.g., leaves).

The average biomass values were determined for four different canopies. The results demonstrated that canopy interior plants performed better than plants in the periphery, as would be expected from the greater transmittance of sunlight. Significantly, an increase in leaf biomass of 10.2% was observed for the canopy interior TLA3-RNAi plants as compared to that of the corresponding wild type plants.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1-21. (canceled)

22. A method of making a Nicotiana tabacum plant, comprising:
selecting a Nicotiana tabacum plant comprising a non-natural mutation in an endogenous nucleic acid sequence encoding a polypeptide having at least 95% sequence identity to a sequence selected from the group consisting of SEQ ID NOs: 22, 24, and 26;
crossing or selfing said selected Nicotiana tabacum plant to produce at least one progeny seed; and
obtaining at least one progeny Nicotiana tabacum plant grown from said at least one progeny seed, wherein said at least one progeny Nicotiana tabacum plant comprises said non-natural mutation; and
wherein said at least one progeny Nicotiana tabacum plant comprising said non-natural mutation has reduced expression or activity of a polypeptide having at least 95% sequence identity to a sequence selected from the group consisting of SEQ ID NOs: 22, 24, and 26 compared to a control tobacco plant lacking said non-natural mutation.

23. The method of claim 22, further comprising:
curing tobacco leaves from said at least one progeny Nicotiana tabacum plant to produce cured tobacco leaves; and
producing a tobacco product from said cured tobacco leaves.

24. The method of claim 23, wherein said tobacco product is selected from the group consisting of cigarettes, smokeless tobacco products, cigarillos, non-ventilated recess filter cigarettes, vented recess filter cigarettes, cigars, snuff, pipe tobacco, cigar tobacco, cigarette tobacco, chewing tobacco, leaf tobacco, shredded tobacco, and cut tobacco.

25. The method of claim 22, wherein said endogenous nucleic acid sequence is selected from the group consisting of SEQ ID NOs: 21, 23, and 25.

26. The method of claim 22, wherein said at least one progeny Nicotiana tabacum plant comprises a reduced amount of mRNA corresponding to SEQ ID NO: 21, 23, or 25, a reduced amount of polypeptide comprising SEQ ID NO: 22, 24, or 26, reduced activity of a polypeptide selected from the group consisting of SEQ ID NOs: 22, 24, or 26; or a combination thereof.

27. The method of claim 22, wherein said at least one progeny Nicotiana tabacum plant comprises a reduced concentration of thylakoid membranes in the photosystems; a reduced amount of total chlorophyll; an increased ratio of chlorophyll a to chlorophyll b;
or a combination thereof wherein said reduced or increased amounts or ratio are relative to a Nicotiana tabacum plant lacking said non-natural mutation.

28. The method of claim 22, wherein leaf from the at least one progeny Nicotiana tabacum plant exhibits comparable or better quality than leaf from a Nicotiana tabacum plant lacking said non-natural mutation.

29. The method of claim 22, wherein said Nicotiana tabacum plant is a Burley type, a dark type, a flue-cured type, or an Oriental type.

30. A Nicotiana tabacum plant, or part thereof, comprising an altered photosynthesis phenotype and reduced expression or activity of a polypeptide having at least 95% identity to a sequence selected from the group consisting of SEQ ID NOs: 22, 24, and 26; wherein said altered photosynthesis phenotype is selected from the group consisting of a reduced concentration of thylakoid membranes in the photosystems, a reduced amount of total chlorophyll, and an increased ratio of chlorophyll a to chlorophyll b; wherein said reduced or increased amounts or ratio are in comparison to a Nicotiana tabacum plant having wild-type activity of said polypeptide.

31. The Nicotiana tabacum plant, or part thereof, of claim 30, wherein said reduced expression comprises a reduced amount of a polynucleotide selected from the group consisting of SEQ ID NOs: 21, 23, and 25 compared to a Nicotiana tabacum plant having a wild-type amount of said polynucleotide.

32. The Nicotiana tabacum plant, or part thereof, of claim 30, wherein leaf from said Nicotiana tabacum plant, or part thereof, exhibits comparable or better quality than leaf from a control Nicotiana tabacum plant lacking said mutation.

33. Cured leaf from the Nicotiana tabacum plant of claim 30.

34. A tobacco product comprising the cured leaf of claim 33.

35. The tobacco product of claim 34, wherein said tobacco product is selected from the group consisting of cigarettes, smokeless tobacco products, cigarillos, non-ventilated recess filter cigarettes, vented recess filter cigarettes, cigars, snuff, pipe tobacco, cigar tobacco, cigarette tobacco, chewing tobacco, leaf tobacco, shredded tobacco, and cut tobacco.

36. A Nicotiana tabacum plant, or part thereof, comprising a non-natural mutation in an endogenous nucleic acid, the wild type endogenous nucleic acid encoding a polypeptide sequence having at least 95% identity to a sequence selected from the group consisting of SEQ ID NOs: 22, 24, and 26; wherein said Nicotiana tabacum plant comprising said non-natural mutation has reduced expression or activity of said polypeptide sequence.

37. The Nicotiana tabacum plant, or part thereof, of claim 36, wherein leaf from said Nicotiana tabacum plant comprises a reduced amount of mRNA corresponding to a sequence selected from the group consisting of SEQ ID NOs: 21, 23, and 25; a reduced amount of a polypeptide selected from the group consisting of SEQ ID NOs: 22, 24, and 26; reduced activity of a polypeptide selected from the group consisting of SEQ ID NOs: 22, 24, and 26; or a combination thereof.

38. The Nicotiana tabacum plant, or part thereof, of claim 36, wherein leaf from said Nicotiana tabacum plant exhibits comparable or better quality compared to leaf from a plant lacking said non-natural mutation.

39. Cured leaf from the Nicotiana tabacum plant of claim 36.

40. A tobacco product comprising the cured leaf of claim 39.

41. The tobacco product of claim 40, wherein said tobacco product is selected from the group consisting of cigarettes, smokeless tobacco products, cigarillos, non-ventilated recess filter cigarettes, vented recess filter cigarettes, cigars, snuff, pipe tobacco, cigar tobacco, cigarette tobacco, chewing tobacco, leaf tobacco, shredded tobacco, and cut tobacco.
